(19) United States
(12) Patent Application Publication
(10) Pub. No.: US 2026/0257011 A1
(43) Pub. Date: Sep. 3, 2026

(54) INFUSION DEVICE WITH AN ORIENTATION SENSOR TEST

(71) Applicant: Phillips-Medisize A/S, Struer (DK)

(72) Inventors: Rune Ravn, Aarhus C (DK); Toke Dupont Koustrup, Bækmarksbro (DK); Kristine Storm Sorensen, Holstebro (DK); Lars Osterballe, Viborg (DK)

(21) Appl. No.: 19/164,586

(22) PCT Filed: May 17, 2024
(86) PCT No.: PCT/EP2024/063739

§ 371 Date: Sep. 12, 2025

(30) Foreign Application Priority Data
May 17, 2023 (EP) 23173919.4

(51) Int. Cl. A61M 5/145; A61M 5/14; A61M 5/36

(52) U.S. Cl. CPC A61M 5/1452; A61M 5/36; A61M 2005/1402; A61M 2205/33; A61M 2205/505

(57) ABSTRACT

An infusion device and a method at an infusion device, wherein the infusion device includes: a receptacle (120) configured to receive a syringe (200) comprising a plunger (202); a plunger driver (104) configured to releasably engage an end of the plunger (202) of the syringe (200) and to move the plunger (202) in a longitudinal direction of the syringe (200); a drivetrain (105), including a motor (106), coupled to move the plunger driver (104); a main controller (112) configured to control travel of the plunger driver (104) in at least a first direction; and an orientation sensor (114), including an accelerometer with a suspended mass, configured to output a first signal including acceleration information and orientation information. The infusion device operatively controls the motor and movement of the plunger driver in accordance with a first determination (402) that a variability of magnitudes of the first signal fulfils a first criterion and a second determination (408) that a mean or median of magnitudes of the first signal fulfil a second criterion.

The invention relates to an infusion device comprising a receptacle configured to receive a syringe comprising a plunger, a plunger driver configured to releasably engage an end of the plunger of the syringe and to move the plunger in a longitudinal direction of the syringe, a drive train, and an orientation sensor configured to monitor a spatial orientation of the infusion device and to continuously output orientation signals containing information about the spatial orientation of the infusion device.

### BACKGROUND

Patients with chronic diseases and conditions may require regular injections of intravenous and/or subcutaneous medicament treatment. For some medicament types, this may also require injection on a regular basis as determined by a treating physician. For certain patients, including younger and less-able patients, care is often provided in a clinical setting. However, this requires patients and caregivers to be present at a treatment location. Many clinics therefore, when possible, educate patients and caregivers so they can administer intravenous (e.g. subcutaneous or intravenous) infusion at home. This reduces burdens on all parties. However, difficulties may arise when self-administering treatment, including determining correct dosages and ensuring the infusion takes place at the appropriate rate. Improvements in existing methods are desired.

Certain treatments involve the infusion of a volume of a reconstituted medicament (i.e., a dried drug medicament restored to its original state by adding liquid). The amount of reconstituted medicament necessary may depend on many factors, including the type of medicament, the condition of the patient, and the type or stage of the disease (or condition). For some diseases, such as e.g. hemophilia, the presence of clotting factor antibodies may also affect the volume of treatment that must be infused. In addition to dosage, intravenous treatments must be administered at a controlled rate, as the capacity of a given vein to receive the drug, in addition to blood flow rate and volume in the vein, is limited. It is therefore possible to infuse an intravenous treatment at an excessive flow rate, particularly if administered via a syringe. This can result in too much drug being infused and can even result in physical damage to the vein. This is particularly true for longer-duration infusions, which require greater control over a longer period of time. Excessive flow rates, at the extreme, can cause a vein to “blow out” or balloon, potentially injuring the patient. Therefore, controlling the flow rate and volume of an infusion is imperative. Moreover, certain drugs, including hemophilia drugs, are prohibitively expensive. Thus, administering the correct amount and at the correct rate reduces the likelihood of wasted product, and thus reduces expense.

A number of infusion steps rely on information about the functioning of the different sensory parts in the infusion device. However, there is a need for an improved method of testing the same.

### SUMMARY

There is provided:

A method at an infusion device, wherein the infusion device includes:
a receptacle (120) configured to receive a syringe (200) comprising a plunger (202);
a plunger driver (104) configured to releasably engage an end of the plunger (202) of the syringe (200) and to move the plunger (202) in a longitudinal direction of the syringe (200);
a drivetrain (105), including a motor (106), coupled to move the plunger driver (104);
a main controller (112) configured to control travel of the plunger driver (104) in at least a first direction;
an orientation sensor (114), including an accelerometer with a suspended mass, configured to output a first signal including acceleration information and orientation information;
   the method comprising:
a first determination (402), at a first time, that a variability of magnitudes of the first signal fulfils a first criterion; wherein the first criterion includes a first threshold;
a second determination (408), at a second time, that magnitudes of the first signal fulfil a second criterion; wherein the second criterion corresponds with a first range of orientations of the infusion device;
at least in accordance with the first determination and the second determination, enabling the main controller (112) to operatively control the motor (106) and movement of the plunger driver (104) at a first period of time.

An advantage is that the plunger driver is enabled to move via the motor, provided the conditions that the motion sensor is working, including that the mass can move at least dynamically when the infusion device is moved, and that the infusion device is handled to assume an orientation corresponding with the first range of directions. These conditions together improve safety associated with use of the infusion device.

In some examples, variability of magnitudes of the first signal is determined over a period of time e.g. less than 500 mS, such as less than 100 mS. In some examples the variability of magnitudes is based on a sum of differences between the magnitudes and a mean value of the magnitudes. In some examples, the variability is a statistical variance. The variability is a measure of the dynamic response of the accelerometer.

In some examples, the first signal includes values corresponding to the instant acceleration acting on the suspended mass. Each value may include a portion caused by movement of the infusion device and a portion caused by the earth's gravitational field. The portion caused by the earth's gravitational field can be observed as an offset associated with the orientation of the infusion device. The portion caused by movement of the infusion device, can be observed as an alternating, dynamic signal superposed on the offset. Orientation of the infusion device can thus be obtained by determining the offset or level shift in one or more directions e.g. in three orthogonal directions in the case of a three-axis accelerometer. Movement of the infusion device can be obtained by determining the variability, e.g. variance, of the first signal.

In some examples, orientation sensor includes a gyroscope. The first signal may include values from the gyroscope. Orientation information may then include explicit orientation information e.g. including values representing an angle of rotation with respect to one or more axes as it is known in the art.

In some examples, the first criterion is fulfilled at times when the infusion device is held, e.g. without fixed support, in a person's hand. Thus, the infusion device and its orientation sensor are exposed to agitation stimuli by means of being hand-held. This is turn causes the first signal to include alternating, dynamic values. The first time may correspond with the user having immediately inserted a battery or pressed a button. The first time may be at a time less than a second after the user having immediately inserted a battery or pressed a button. This reasonably guarantees that the infusion device is held-held at the first time.

In some examples, the second criterion includes a threshold or range of mean or median magnitude values. In some examples, the threshold or range of mean or median magnitude values enables detecting that the infusion device assumes a first spatial orientation (e.g. 90 degrees corresponding to ‘up’) or an orientation (e.g. between 60 and 120 degrees) about the first spatial orientation. The second criterion can thus define a range of orientations of the infusion device. Determining the offset or level shift in values in the first signal can be used to determine the orientation of the infusion device. Orientation of the infusion device can thus be obtained by determining the offset or level shift in one or more directions e.g. in three orthogonal directions. Whereas the (faster) alternating, dynamic portion of the values are associated with shaking of the device, the (slower) offsets or level shifts are associated with an orientation of the device. A filter may be implemented to provide disambiguation between determining shaking (more dynamic) and orientation (more static).

In some examples, the second criterion is fulfilled at times when the infusion device is held within a first range of orientations. In some examples, the first range of directions corresponds to an orientation of the syringe, with its outlet pointing upwards, when accommodated in the receptacle in the infusion device.

One or more of the receptacle, the plunger, the plunger driver, the drivetrain, the motor, the main controller, and the orientation sensor is/are accommodated in a housing.

In some examples, the infusion device is a battery-operated infusion device also known as an internally powered infusion device.

In some embodiments the first signal output by the orientation sensor is associated with a noise floor level and a maximum magnitude; and wherein the first threshold is at a value above the noise floor level and below the maximum magnitude.

An advantage is that the first threshold enables detecting that the suspended mass moves physically when subjected to a movement e.g. an agitation of the infusion device, e.g. by being hand-held without a fixed support. At least since the first threshold is above the noise floor level, a physical movement of the infusion device is required to exceed the first threshold. Thus, if the first threshold is exceeded, it can be concluded that the suspended mass is movable and not stuck.

In some examples, the second determination includes that a mean or median of magnitudes of the first signal fulfil a second criterion. This enables disambiguation of the orientation information.

In some examples, the first threshold is set in conjunction with a period of time over which the mean or median is determined to enable that only a movement, rather than noise, is likely to satisfy the second criterion.

In some examples, the first threshold is at a value below or above a magnitude corresponding to a gravitational acceleration.

In some embodiments the method comprises:
a third determination (404), at a third time, that magnitudes of the first signal fulfil a third criterion; wherein the third criterion corresponds with a second range of orientations of the infusion device; wherein the second range of orientations is different from the first range of orientations; wherein the third time is at or prior to the second time and at or after the first time; and
at least in accordance with the third determination, enabling the main controller (112) to operatively control movement of the motor (106) and movement of the plunger driver at the first period of time.

An advantage is that testing of the orientation sensor is further improved by testing that the suspended mass can move correctly corresponding to the infusion device having different orientations and that correct handling of the infusion device can be tested as well e.g. handling including a rotation of the infusion device.

In some examples, the third time precedes the second time. In some examples, the first time precedes the third time.

In some examples, the second range of orientations correspond to an orientation of the syringe, with its tip or outlet pointing upwards (opposed to downwards), when accommodated in the receptacle in the infusion device. Correspondingly, the first range of directions corresponds to an orientation of the syringe, with its tip or outlet pointing downwards (opposed to upwards), when accommodated in the receptacle in the infusion device.

In some embodiments the method comprises:
forgoing performing the second determination (407), in accordance with a determination that the second time does not fall within a first time period running from the third time.

An advantage is that it can be tested that the user correctly turned the infusion device prior to and as a condition for operating the motor and thus the plunger driver.

The first time period is longer than about 10 seconds, e.g. longer than about 20 seconds, longer than about 30 seconds or longer than about 40 seconds.

In some embodiments the method comprises:
in accordance with a failure to detect, using one or more detectors at the infusion device, at a time preceding the first time, that a user is handling the infusion device, forgoing performing the first determination (401).

An advantage is a reliable test of the dynamic capabilities of the accelerometer requiring a real handling movement of the infusion device preceding a user interaction detectable via the one or more detectors.

Thus, if the user is not detectably and likely handling the infusion device at a time in extension of a user intervention including the first time, the accelerometer is not tested at this time to verify its dynamic capabilities. In some examples, the main controller forgoes enabling operatively controlling the motor at least until a time when the second determination is made.

The one or more detectors may include a power-on detector, a button or switch e.g. at the user interface, and/or another detector capable of detecting an event that can be associated with a user currently handling the infusion device. The one or more detectors at the infusion device may be different from or include the orientation sensor.

In some embodiments one or both of the second determination (408) and the third determination (404) includes that a mean or median of magnitudes of the first signal fulfil the second criterion; wherein the mean or median of magnitudes of the first signal is determined over a period of time being shorter than about 500 milliseconds, e.g. shorter than about 300 milliseconds, e.g. about 100 milliseconds.

At least in some examples, the orientation information is embedded in the acceleration information. So, the orientation information may be derived from the acceleration information. In this respect, an advantage is a useful disambiguation of orientation information by suppressing alternating variations in the values included in the first signal. The period of time may coincide with or precede, such as immediately precede the second time (at which the second determination is made). The disambiguation may include non-linear filtering or, alternatively, linear filtering. In some examples, the method provides disambiguation of orientation information by filtering out portions of the first signal alternating above a cut-off frequency e.g. a cut-off frequency at about 1 Hz.

In some embodiments the method comprises:
in accordance with a failure to determine that the first criterion is fulfilled at the first time and/or a failure to determine that the second criterion is fulfilled at the at the second time:
forgoing enabling the main controller (112) to operatively control the motor (106) and movement of the plunger driver at the first period of time; and
initiating a first exception procedure (410).

An advantage is that a malfunction of the electromechanical motion sensor can be handled by the first exception procedure and that any damage which may have been caused by incorrect movement of the plunger driver can be prevented e.g. if the orientation sensor is damaged in a way that the movable mass is not able to move.

In some examples, the first exception procedure includes guiding a user via a user interface at the infusion device to correct handling of the infusion device. The exception procedure generally forgoes enabling operatively controlling the motor. The exception procedure may include one or more retry-procedures enabling exiting the exception procedure upon successful completion of one or more of the first, second and third determination(s).

In some embodiments the method comprises:
in accordance with a failure to determine that the third criterion is fulfilled at the third time:
forgoing enabling the main controller (112) to operatively control the motor (106) and movement of the plunger driver at the first period of time; and
initiating the first exception procedure (410).

An advantage is a further check the that the motion sensor is working, including that the mass can move, due to gravitational forces, in response to the infusion device being handled, including turned.

In some examples, the second criterion is fulfilled when the infusion device has a first orientation, e.g. pointing downwards, and the third criterion is fulfilled when the infusion device has a second orientation, e.g. pointing upwards.

In some embodiments the exception procedure includes:
in accordance with a failure to make the first determination (402), emitting a first message; wherein the first message prompts a user to restart the infusion device;
in accordance with a failure to make the second determination (408) and/or a failure to make the third determination (404), emitting a second message; wherein the second message guides a user to change orientation of the infusion device.

An advantage is the disambiguation of situations wherein the user can change handling of the infusion device to proceed to enabling the main controller to operatively control the motor and movement of the plunger driver. This assists the user in operating the infusion device to proceed to or towards an infusion step.

In some examples, the exception procedure maintains a counter counting the number of times each of one or more of the first determination, the second determination and the third determination fails. The exception procedure may forgo or change the first message and/or the second message in response to reaching or exceeding a counter value.

The first message and the second message may be a message displayed on a display at the infusion device, e.g. a display of a user interface at the infusion device. In some examples, the first message and/or the second message may include a sound generated by a sound generator or a haptic response generated by a haptic actuator.

In some embodiments the infusion device is configured to receive a battery for supplying battery power to the main controller; the method comprising:
detecting a first event including a battery-power-on event; and
in response to detecting the first event, and a failure to make at least the first determination, initiating the first exception procedure.

An advantage is that the first exception procedure can be initiated immediately, and without further user interaction, in response to the failure to make the first determination. Thus, in response to a battery-power-on event, the accelerometer can be tested without further user input.

In some embodiments the infusion device includes a first input element connected to the main controller for receiving a user input at the input element; the method comprising:
detecting a second event including a first input at the first input element;
in response to detecting the second event, and a failure to make at least the second determination (408) and/or a failure to make at least the third determination (404):
forgoing enabling the main controller (112) to operatively control the motor (106) and movement of the plunger driver at the first period of time; and
initiating the first exception procedure (410).

An advantage is that testing of the accelerometer and that the suspended mass can be moved can be timed with a user interaction including the user input at the input element and thus a strong expectation that the user is currently handling the infusion device.

In some embodiments the infusion device includes a first output element connected to the controller; wherein the output unit includes one or more of: a display unit, a sound actuator, and a haptic actuator; the method comprising:
detecting the second event in response to generating an output via the output element.

An advantage is that testing of the accelerometer is improved by requiring that the user interaction including the user input at the input element is a timely response to an output via the output element. A user may be prompted by a display message on the display, by sound or by a haptic signal, to give an input via the input element.

A haptic actuator create motion, e.g. vibration, that allow electronic devices to impart information to the user through the user's sense of touch.

In some embodiments the method comprises:
in response to detecting the first event and/or the second event, and a failure to make one or more of the first determination (402), the second determination (408), and the third determination (404):
initiating the first exception procedure (410).

An advantage is that both of dynamic and static capabilities of the accelerometer is tested in response to detection of the battery-power-on event. If one or both of the dynamic and static capabilities fails to pass, the exception procedure can be initiated immediately without further user interaction.

In some embodiments the method comprises:
in response to detecting the second event, and a failure to make at least the third determination (404):
forgoing enabling the main controller (112) to operatively control the motor (106) and movement of the plunger driver at the first period of time; and
initiating the first exception procedure (410).

An advantage is that the user's correct handling of the infusion device is required as a condition for enabling the motor to move the plunge driver and thus the plunger.

In some embodiments the infusion device includes a display unit; the method comprising:
in response to the second determination (408), corresponding with a first range of orientations of the infusion device, displaying a first display image on the display in accordance with a first display orientation;
in response to the third determination (404), corresponding with a second range of orientations of the infusion device, displaying a second display image on the display in accordance with a second display orientation.

An advantage is that the controller uses the signal from the orientation sensor to rotate information, such as text and icons, on the graphical display depending on the orientation of the infusion device. This can improve readability of the information including instructions for the use of the infusion device.

The display image may include one or more of the following: text, icons, and images. The display image represents the content of what is displayed on the display e.g. all content or a portion of all content. The content of the display image may depend on a current status of the infusion device e.g. including the orientation of the infusion device. In some examples, the first display image and the second display image are different. In some examples, the first display image and the second display image are identical or substantially identical.

As stated above, the third determination, at the third time, includes that absolute magnitudes of the first signal fulfil a third criterion; wherein the third criterion corresponds with a second range of orientations of the infusion device; wherein the second range of orientations is different from the first range of orientations; wherein the third time is at or prior to the second time and at or after the first time.

In some embodiments the method comprises:
at the third time, communicating a second message via the first output element and starting a timer with a second time period; wherein the third time is at or prior to the second time and at or after the first time;
in accordance with determination that the second determination is made before the second time period lapses, enabling the main controller (112) to operatively control the motor (106) and movement of the plunger driver at the first period of time;
in accordance with determination that the second determination cannot be made before the second time period lapses, communicating a third message via the output element and forgoing enabling the controller (112) to operatively control the motor (106) and movement of the plunger driver at the first period of time.

An advantage is the further condition for enabling movement of the motor. In some examples, the second time period is longer than about 10 seconds, longer than about 20 seconds, or longer than about 30 seconds.

In some examples, the second message includes instructions to a user to rotate the infusion device. In some examples, the third message includes instructions to a user to reset or restart the infusion device.

In some embodiments the method comprises:
at the first period of time, at a time when the controller is enabled to operatively control the motor and the movement of the plunger driver, performing a priming step including operating the motor to enact that the plunger driver (104) engages the end of the plunger (202) of the syringe (200) and moves the plunger (202) in a longitudinal direction of the syringe (200); wherein at least a volume of air is expelled from the syringe;
   wherein the first range of orientations of the infusion device corresponds with an outlet of the syringe pointing upwards.

In some examples, the syringe points upwards at least in an orientation that is substantially vertical with the outlet pointing upwards or in an orientation that is closer to a vertical orientation than to a horizontal orientation with the outlet pointing upwards or in an orientation that is inclined but with the outlet pointing in a direction above the horizontal plane.

There is also provided an infusion device comprising:
a receptacle (120) configured to receive a syringe (200) comprising a plunger (202);
a plunger driver (104) configured to releasably engage an end of the plunger (202) of the syringe (200) and to move the plunger (202) in a longitudinal direction of the syringe (200);
a drivetrain (105), including a motor (106), coupled to move the plunger driver (104);
a controller (112) configured to control travel of the plunger driver (104) in at least a first direction;
an orientation sensor (114), including an accelerometer with a suspended mass, configured to output a first signal including acceleration information and direction information;
   wherein the infusion device is configured to perform the method set out in any of the embodiments above.

Advantages and aspects thereof are described above.

In some examples, the orientation sensor includes a micro electro-mechanical system (MEMS), such as a 3-axes accelerometer e.g. including a gyroscope.

In some embodiments the infusion device comprises one or more of:
a first input element connected to the controller for receiving a user input at the input element; and
an output element connected to the controller; wherein the output unit includes one or more of: a display unit, a sound actuator, and a haptic actuator.

In some examples, the display unit is a graphical display, e.g. a touch-sensitive display including the first input element.

In some embodiments the infusion device is configured to receive a battery; wherein the battery can supply battery power to the controller.

### BRIEF DESCRIPTION OF THE FIGURES

Various examples are described hereinafter with reference to the figures. Like reference numerals refer to like elements throughout. Like elements will, thus, not be described in detail with respect to the description of each figure. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

FIGS. 1A and 1B show an example of an infusion device according to an example of the invention;

FIG. 1C shows a housing of the infusion device;

FIGS. 1E-H show some of the internal parts of the infusion device shown in FIGS. 1A and 1B;

FIG. 1I shows a schematic overview of the relation between some of the internal parts of the infusion device shown in FIGS. 1A and 1B;

FIG. 2 shows an example of an evaluation of the orientation sensor in different spatial orientations;

FIG. 3 shows a flowchart for a method at the infusion device; and

FIG. 4 shows examples of display images and display updates.

### DETAILED DESCRIPTION

Exemplary examples will now be described more fully hereinafter with reference to the accompanying drawings. In this regard, the present examples may have different forms and should not be construed as being limited to the descriptions set forth herein. Accordingly, the examples are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The spatially relative terms “lower” or “bottom” and “upper” or “top”, “below”, “beneath”, “less”, “above”, and the like, may be used herein for ease of description to describe the relationship between one element or component and another element or component as illustrated in the drawings. It will be understood that the spatially relative terms are intended to encompass different orientations of the device in use or operation, in addition to the orientation depicted in the drawings. For example, in the case where a device illustrated in the drawings is turned over, elements described as being on the “lower” side of other elements, or “below” or “beneath” another element would then be oriented on “upper” sides of the other elements, or “above” another element. Accordingly, the illustrative term “below” or “beneath” may include both the “lower” and “upper” orientation positions, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below, and thus the spatially relative terms may be interpreted differently depending on the orientations described.

Throughout the specification, when an element is referred to as being “connected” to another element, the element is “directly connected” to the other element, or “electrically connected” to the other element with one or more intervening elements interposed therebetween.

The terminology used herein is for the purpose of describing particular examples only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that, although the terms “first,” “second,” “third,” and the like may be used herein to describe various elements, these elements should not be limited by these terms. These terms are only used to distinguish one element from another element. Thus, “a first element” discussed below could be termed “a second element” or “a third element,” and “a second element” and “a third element” may be termed likewise without departing from the teachings herein.

About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

Exemplary examples are described herein with reference to cross section illustrations that are schematic illustrations of idealized examples, wherein like reference numerals refer to like elements throughout the specification. As such, variations from the shapes of the illustrations as a result, for example, of manufacturing techniques and/or tolerances, are to be expected. Thus, examples described herein should not be construed as limited to the particular shapes of regions as illustrated herein but are to include deviations in shapes that result, for example, from manufacturing. For example, a region illustrated or described as flat may have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims. Some of the parts which are not associated with the description may not be provided in order to specifically describe exemplary examples of the present disclosure.

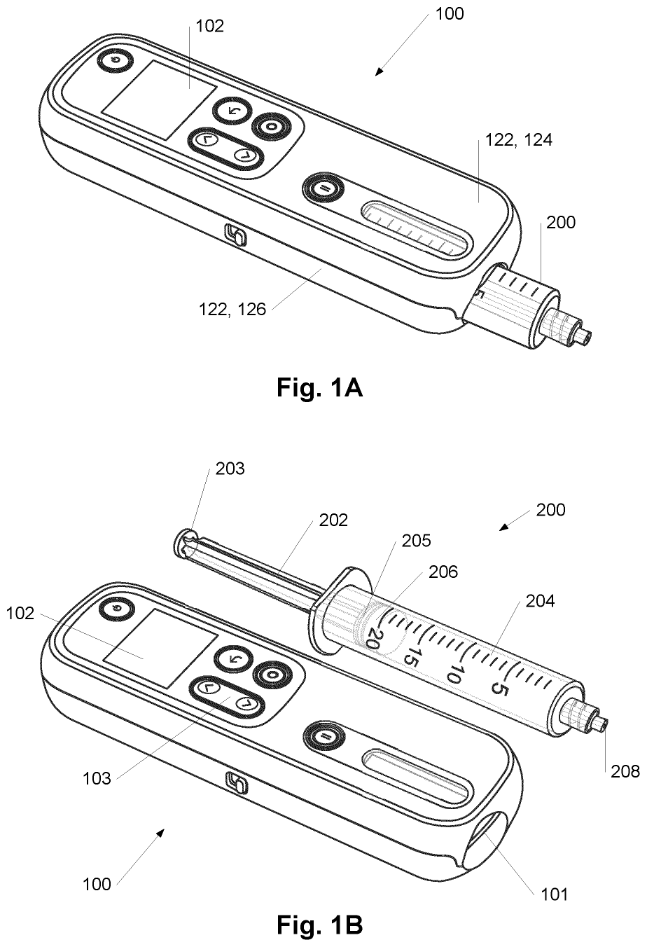

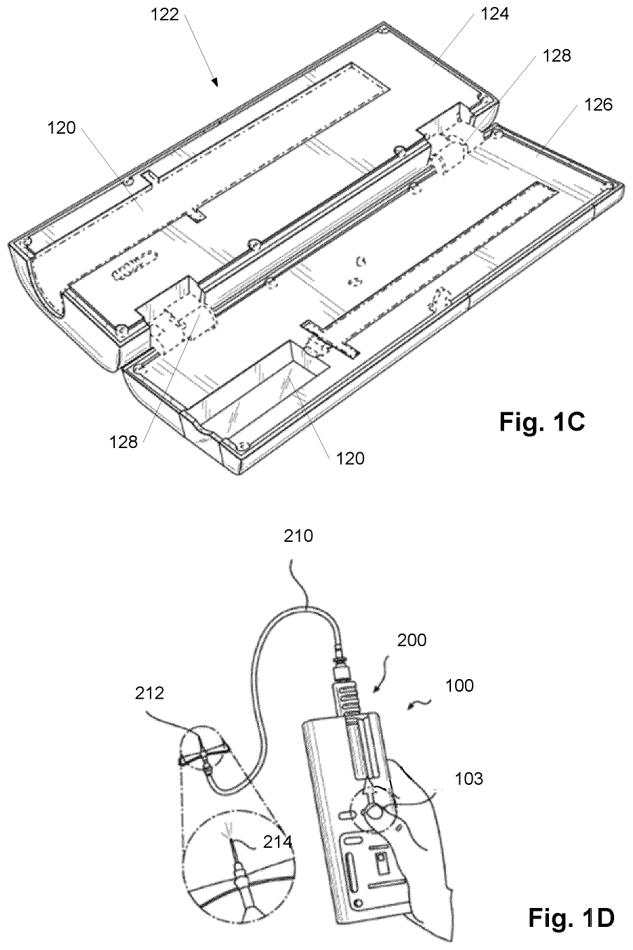

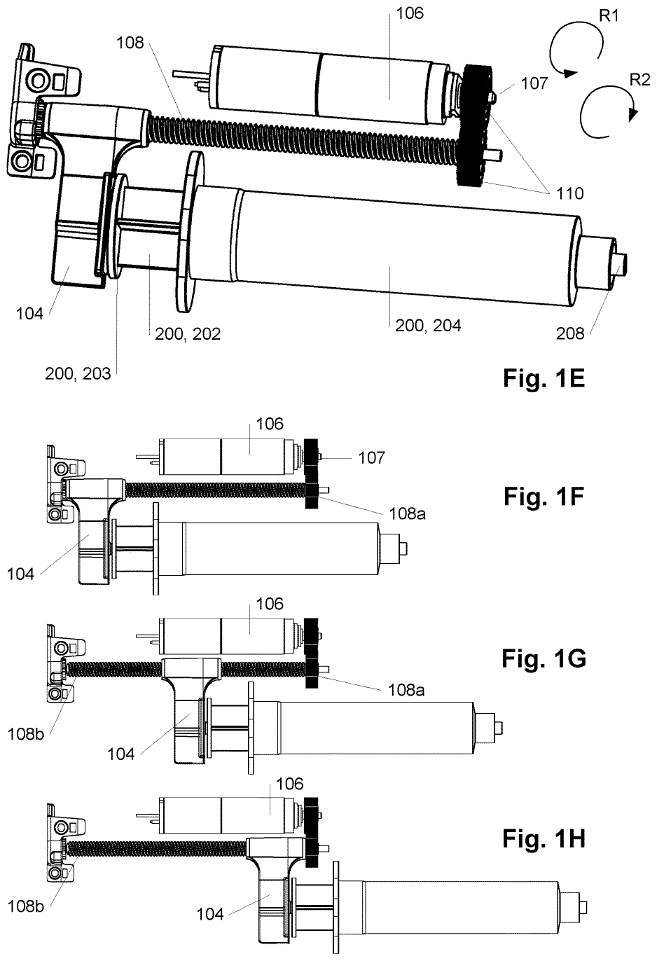

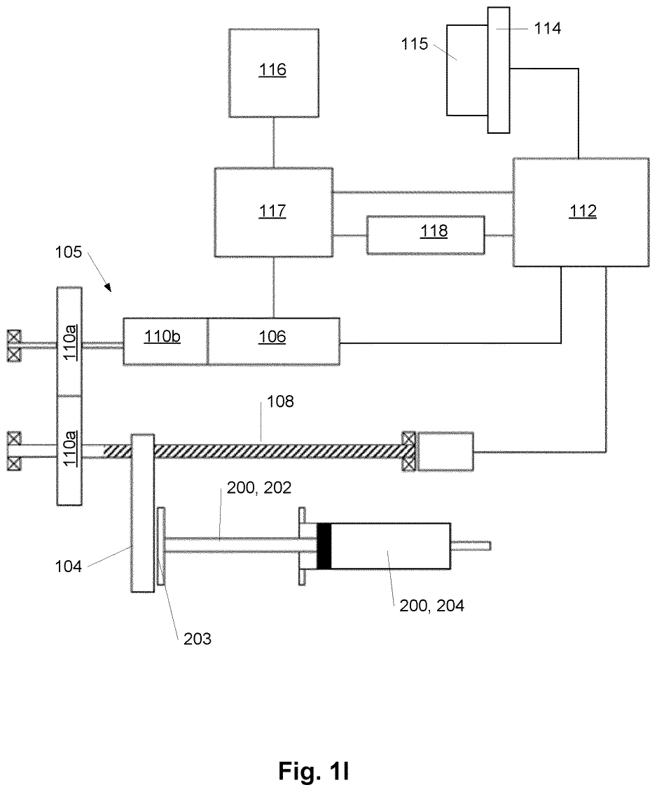

FIGS. 1A and 1B show an example of an infusion device 100 with a syringe 200 comprising a plunger 202 according to an example of the invention. FIG. 1B shows the infusion device 100 and the syringe 200 side by side, whereas in FIG. 1A, the syringe 200 is arranged within the infusion device 100 so that the plunger 202 cannot be seen in this figure. The syringe 200 may be supported within the receptacle 120 shown in FIG. 1C such that an end of syringe 200 and/or an infusion line coupled to the syringe 200 extends through a distal end of the infusion device 100. The syringe 200 supported in the receptacle 120 is shown in FIG. 1A and FIG. 1D, where FIG. 1D shows an infusion line 210 coupled to the syringe 200.

In one or more examples, the syringe 200 may be a generic, over-the-counter syringe comprising a plunger 202 that is movable, e.g., by hand, to administer fluids and/or a treatment agent (e.g., medication or drug, such as, for example, a reconstituted drug) for example subcutaneously or intravenously. In other examples, the syringe 200 may be a specially-designed syringe, e.g., that is specifically suited for administering a treatment agent. Further, the syringe 200 may be prefilled with medication, i.e. not needing a syringe filling application where typically the user fills the syringe by withdrawal of medicament/drug from a primary container vial.

The infusion device 100 can, e.g., be worn by a patient. The infusion device 100 may be coupled to an infusion line 210 as shown in FIG. 1D. The infusion line 210 is coupled to a needle 212 with a needle outlet 214 through which the medicament exits the syringe infusion line 210.

The infusion device 100 as shown in FIGS. 1A-B comprises a housing 122 inside which is found a receptacle 120 for receiving the syringe 200. The receptacle 120 may thus be configured to support the syringe 200 and to control the syringe 200 for the administration of medicament into and from the syringe 200. An example of the housing 122 and the receptacle 120 is shown in FIG. 1C showing an open empty housing 122. The housing 122 comprises a housing top part 124 and a housing bottom part 126. The housing top part 124 of the infusion device 100 is movable with respect to the housing bottom part 126 such that access to the interior of the infusion device 100 is possible for mounting of a syringe 200 inside the infusion device 100. The housing top part 124 may secure over an upper edge of the housing bottom part 126 and may form an interior volume of the infusion device 100 when in a closed state. The housing top part 124 may be connected to the housing bottom part 126 by one or more hinges 128, allowing the housing top part 124 to rotate relative to the housing bottom part 126 when transitioning between an open state and the closed state. Together with the hinges 128, one or more latches or connectors may also be disposed along an edge of the housing top part 124 or the bottom housing part 126 to secure the housing top part 124 to the housing bottom part 126. The one or more latches may be disposed on an edge of the housing bottom part 126 and/or the housing top part 124 that are not shared with the one or more the hinges 128. The housing top part 124 may be preferably opened by pivoting about the hinges 128, revealing the interior volume of the infusion device 100 when inserting syringe 200 into or removing syringe 200 from the infusion device 100. The receptacle 120 may preferably extend along a substantial portion of the infusion device 100 such that a length of the receptacle 120 may be configured to accommodate syringe 200 and the plunger 202 when the plunger 202 is fully extended and positioned within the receptacle 120. However, it is also contemplated that the receptacle 120 may be formed merely by the syringe opening 101 in the infusion device 100, wherein the syringe opening 101 alone may support the syringe 200.

On the outside of the housing top part 124 (see FIGS. 1A-B) is a user interface 102 configured to allow a user, such as a health care person, to enter e.g. codes and to access and adjust settings of a controller 112 of the infusion device 100 (see FIG. 1I). In addition to the user interface 102, a set of plunger moving controllers 103 are seen in FIGS. 1A-B. The plunger moving controllers 103 may be configured for controlling the movement of a plunger driver 104 (see FIGS. 1E-I) inside the infusion device 100.

The infusion device 100 comprises a number of internal parts inside the infusion device 100 as shown in FIGS. 1E-H. In FIG. 1E, it is seen how the plunger driver 104 of the infusion device 100 interacts with the plunger 202 of the syringe 200, the latter which is in contact with a medicament compartment 204 of the syringe 200. The plunger driver 104 is configured to releasably engage a driver end 203 of the plunger 202 and to move the plunger 202 in a longitudinal direction of the syringe 200. The medicament compartment 204 may have a generally cylindrical shape, although other shapes are also contemplated. A portion of the medicament compartment 204 may be disposed within the infusion device 100 while a remaining portion of the medicament compartment 204 may project out through a syringe opening 101. The plunger 202 may be slidably received within the medicament compartment 204. For example, the medicament compartment 204 may include a generally hollow and elongated enclosure and the plunger 202 may be slidingly disposed in the enclosure within the medicament compartment 204. The plunger 202 may extend from the driver end 203 disposed outside the medicament compartment 204 to a medicament end 205 disposed within the medicament compartment 204. The medicament end 205 of the plunger 202 may be in contact with a movable seal 206 with the medicament compartment 204.

In addition to having a plunger rod mounted on the syringe, the syringe may optionally be mounted without a plunger rod, whereby the plunger (or a piston) in the pump instead is shaped to allow for pressing into the syringe body during delivery of the medicament/drug to the patient. Using a syringe without a plunger rod may allow for other syringe mountings in the pump by axially loading the syringe from e.g. the front end of the pump (not shown in the figures).

FIG. 1E also illustrates how the plunger driver 104 is driven by drive train 105 (cf. FIG. 1I) comprising a rotating motor 106, such as a DC motor, via a lead screw 108, which is connected to the motor 106 through a gearing arrangement comprising one or more gears 110.

The plunger interface between the plunger driver 104 and the plunger 202 may be configured as a ‘push only’ interface moving in a forward longitudinal direction for emptying the syringe as shown in three stages in FIGS. 1F-H. However, the plunger interface may also be implemented for both pushing and pulling, hence allowing a pump to support aspiration (needle in blood vessel) and/or filling the syringe from an external vial primary container. Thus, the lead screw 108 may be configured to engage the driver end 203 of the plunger 202 to move the plunger 202 in a first direction. The lead screw 108 may also be configured to move the plunger 202 in a second direction, opposite the first direction. The lead screw 108 may be configured to engage with and/or grasp the driver end 203 of the plunger 202 when the driver end 203 is located adjacent or close to the lead screw 108. The gripping of the driver end 203 may be obtained by the lead screw 108 being connected to or equipped with a grip arrangement (not shown) gripping the driver end 203 of the plunger.

The plunger moving controllers 103 as shown in FIGS. 1A-B, allow the user to provide input to the infusion device 100 and thereby control the movement of the plunger 202 of the syringe 200 when the syringe 200 is supported in the infusion device 100. The plunger moving controllers 103 may comprise two push buttons, one for each opposite direction, which, when pressed, may provide inputs to the controller 112 (see FIG. 1I) of the infusion device 100 to control the movement of the plunger 202 in the first direction and/or in the second direction. The placement of the plunger moving controllers 103 around a middle region of the infusion may allow a user to grip the infusion device 100 with one hand and make inputs to the plunger moving controllers 103 with the user's thumb. As an alternative to the two plunger moving controllers 103 shown in FIG. 1A-B, a joystick capable of moving and/or tilting in two opposite directions for moving the plunger 202 in the first direction and/or in the second direction may also be envisioned.

The user interface 102 may include a digital display and/or touch screen display. As an alternative or in addition to the user interface 102 and the plunger moving controllers 103 shown in FIG. 1A-B, additional user controls may also be envisioned. These may include buttons, switches, or any other interface that is configured to receive input from a user and provide signals to the controller 112 of the infusion device 100. In some examples, user controls may comprise mechanical and/or electronic interfaces that can receive inputs from the touch of a user or a motion imparted by the user and provide signals to the controller 112 of the infusion device 100. Exemplary electronic interfaces include resistive touch, capacitive, or equivalent interfaces. Further still, user controls may be integrated into the user interface 102 by way of a touchscreen display. User controls and the user interface 102 may be preferably disposed on the outside of the infusion device 100.

FIG. 1I shows a schematic overview of the relation between the internal parts 105, 104, 106, 108, 110, 112 of the infusion device. In FIG. 1I, the main controller 112 is also shown. The gearing arrangement comprising one or more gears 110 may comprise a planetary gear 110b and two gear wheels 110a as shown in FIG. 1I. Also shown in FIG. 1I is an orientation sensor 114, an orientation sensor self-test system 115, a battery 116 powering the infusion device 100, a driver 117, and a current measuring sensor 118.

The motor 106 may drive the lead screw 108. The motor 106 may be controlled by the plunger moving controllers 103, user controls, and/or one or more signals generated from a control algorithm stored in the main controller 112.

The drive train 105 comprises the lead screw 108 that may be configured to be rotated by the motor 106, causing the lead screw 108 to translate in the first direction or in the second direction based on a direction of rotation of the lead screw 108. The lead screw 108 may preferably extend along a length of the infusion device 100 such that the position of the lead screw 108 along the length of the receptacle 120 is controllable. A length of travel of the lead screw 108 may be determined by a length of the lead screw 108. The lead screw 108 may be configured to engage the driver end 203 of the plunger 202 when the plunger 202 is fully extended from or fully inserted into the medicament compartment 204, and anywhere in between. Thus, the lead screw 108 may be configured to engage an extended plunger 202 and drive the plunger 202 completely into the syringe compartment 204 of syringe 200 to administer and/or otherwise evacuate the entire contents of syringe 200. Similarly, the lead screw 108 may be configured to engage the plunger 202 regardless of how far the plunger 202 is inserted into the medicament compartment 204, and to retract the plunger 202 in the syringe compartment 204 of syringe 200 to load and/or otherwise fill syringe 200.

The motor 106 of the infusion device 100 may receive inputs from the plunger moving controllers 103 and may either directly drive the lead screw 108 or indirectly drive the lead screw 108 via the drive train 105.

The drive train 105 may include one or more gears 110 to reduce or increase the rotational speed and torque of the motor 106 on the lead screw 108. The relative size of each gear may vary based on a desired gear reduction or amplification between the motor 106 and the lead screw 108, and the anticipated load being imparted on the gear.

The lead screw 108 may preferably include a threaded outer surface having a thread pitch and a thread direction. The lead screw 108 may be coupled to the gears 110, whereby rotation of the gears 110 may cause the threads of the lead screw 108 to impart a translation on the lead screw 108 along a length of the lead screw 108.

The motor 106, imparting a rotation on the lead screw 108, may control the position of the lead screw 108 by controlling a direction of rotation and rotational velocity of the lead screw 108. The plunger moving controllers 103 may send signals to the motor 106 to control the direction of travel and speed of plunger driver 104. The plunger moving controllers 103 may also control the lead screw 108 when it attaches to the plunger 202 and when the lead screw 108 is otherwise being moved or positioned within the interior of the infusion device 100.

During an infusion procedure, the controller 112 may utilize inputs from the plunger moving controllers 103, or in other examples may only receive inputs from user controls and stored algorithms in a memory. For example, it may be desirable to control an infusion using a predefined infusion algorithm stored in the memory rather than allowing a user to control the speed at which fluid and treatment agent is administered. For example, certain treatment agents may require a particular infusion rate based on different variables. These variables may relate to characteristics of the user (e.g., height, weight, blood diagnostics, etc.), and they may relate to the type of fluid or treatment agent being infused. Therefore, it may be desirable to carefully control the infusion rate without allowing the user to interfere, for instance, by speeding up or slowing down an infusion. In this situation, a stored algorithm in the memory may be executed by the controller 112, whereby the controller 112 may carry out specific sequences by sending signals to the plunger driver 104 to control the movement of the lead screw 108 and thus movement of syringe the plunger 202. The particular algorithm selected for an infusion and the variables serving as inputs for the algorithm may be loaded in various ways into the memory or provided to the controller 112. For instance, user controls and/or the user interface 102 may allow a user to manually input characteristics necessary to determine the infusion rate (e.g., by selecting the treatment agent to be infused, entering information about the user, etc.). The user interface 102 may prompt the user for necessary inputs and either the display itself (e.g., a touchscreen display) may be used or user controls may be utilized to respond to the prompts before the infusion begins. Alternatively, the infusion variables for the infusion algorithm may be preloaded or loaded using an alternative data transfer protocol that does not rely on manual input using the infusion device 100.

For instance, the infusion device 100 may include a serial interface, near-field communications (e.g., RFID), BlueTooth, Wi-Fi, or equivalent wired or wireless data transfer protocol for transferring information relating to algorithms that may be stored in the memory for execution by the controller 112. In some examples, the infusion device 100 may have an NFC reader capable of wirelessly reading the infusion variables or other information from a label or other description on a vial containing a fluid and/or a treatment agent. A third-party device, such as a smartphone or PC computer may also be used to prepare the algorithm and enter the necessary variables, after which the algorithm may be transferred to the infusion device 100 for execution by the controller 112. It is understood that a combination of inputs may be utilized to select and configure an infusion algorithm used to carry out the infusion. The algorithm may also operate completely independently of user input during execution (e.g., from input via the plunger moving controllers 103 or user controls), or the controller 112 may accept and process inputs from the user to alter, stop, or initiate the infusion algorithm.

For examples in which inputs may alter the infusion algorithm, user controls, such as buttons, switches, or a touchscreen interface, may be used to, among other things, increase the infusion rate, decrease the infusion rate, start the infusion, or cease the infusion. Alternatively, the inputs may be provided using the plunger moving controllers 103. During an infusion, the user may also receive feedback from the infusion device 100 in the form of visual, audible, and/or tactile responses. In one or more examples, the user interface 102 may provide the user with visual information about the infusion setup and its progress, or may provide any other indication necessary during, prior to, or after an infusion. For example, the user interface 102 may display images, icons, and/or text for providing instructions to the user regarding use of the infusion device 100. In addition, the user interface 102 may indicate a current status of the infusion or provide instructions for operating the plunger moving controllers 103 prior to, during, or after an infusion. Further still, these functions may be provided using an indicator, which may include an audible noise generator, a sound generator, a tactile feedback device such as a vibratory device, or equivalent device providing auditory or tactile feedback for a user.

The orientation sensor 114 is configured to monitor a spatial orientation of the infusion device 100 and to continuously output orientation signals containing information about the spatial orientation of the infusion device 100.

Prior to an infusion, a user may prime the infusion line 210 with a fluid and/or treatment agent that will be administered during the infusion procedure. This may involve clearing the infusion line 210 of any air before inserting the needle 212 into a vein. Generally, this process may be conducted by pressing on the plunger 202 of syringe 200 while inverting syringe 200, thereby forcing pockets of air to move to the end of the infusion line 210 and to be expelled from the needle 212. The plunger 202 may be depressed into the syringe compartment 204 of syringe 200, causing the air to evacuate and the fluid/treatment agent to advance to the end of the infusion line 210 and/or the needle 212. This may prevent air from being injected into an individual's vein. Once all air has been evacuated, the needle 212 can be inserted into a vein. The orientation sensor 114 may be configured to display the orientation of the infusion device on the user interface 102 and subsequently instruct the user to perform a priming process, where air is evacuated from the syringe compartment 204. During a priming process, air-bubble in the syringe compartment 204 must be closest to a syringe outlet 208 of the syringe compartment 204 for it to be evacuated without a loss of medicament. Hence, due to gravity, the syringe outlet 208 of the syringe compartment 204 should point substantially upwards, e.g. up to 20 degrees, such as up to 45 degrees from straight vertical direction at which point the plunger 104 movement can be initiated.

When clearing the infusion line 210 of air or when aspirating blood from a vein, the user interface 102 may provide the user with an indication of when to begin and when to end operation of the plunger moving controllers 103 so as not to expel any treatment agent from the infusion line 210 or draw too much blood from the vein. In some examples when clearing the infusion line 210 of air, an indicator may provide a series of audible tones at a given pitch, frequency, duration, or magnitude. The tones may change depending on an amount of force applied to the plunger moving controllers 103 by the user, indicating how fast the lead screw 108 is moving and how fast the plunger 202 is being driven. In one or more examples, an audible or tactile signal may be generated that changes in at least one of frequency, pitch, duration, and/or magnitude proportionally to the force applied to the plunger moving controllers 103. This may be particularly useful when clearing air from infusion line. The user may be provided with feedback as to how fast air is being expelled so that the user can slow down the lead screw 108 before any treatment agent and/or fluid is expelled from infusion line. For treatment agent or fluids that are particularly expensive and/or scarce, this may prevent spillage and may allow the user to predict when to stop applying force to the plunger moving controllers 103 when clearing air from infusion line prior to an infusion. Examples of such expensive treatment may include hemophilia treatment agents, which are generally administered in small doses and the contents of which are very expensive.

The orientation sensor 114 may be an accelerometer configured to determine an orientation of the infusion device 100 relative to the gravitational direction. By spatial orientation is included an orientation in three dimensions, such as an orientation in an orthogonal X, Y, Z axes dimensional space. The orientation sensor may be a micro electro-mechanical system MEMS (micromachined component with an internally suspended mass. In some examples, the orientation of the accelerometer is mapped to the spatial orientation of the infusion device to convert or transform three accelerations signals, corresponding with three orthogonal directions with reference to the accelerometer, to three orthogonal directions with reference to the infusion device.

The orientation sensor 114 may provide inputs to the controller 112, which may display the orientation of the infusion device on the user interface 102.

A MEMS orientation sensor may be sensitive to high-level impacts, such as for example a drop to the floor. In rare situations, the micromechanical subsystem of a MEMS sensor has been known to be damaged. This entails that the vibrating portion becomes stuck fixedly and hence may no longer move/vibrate in response to outer component acceleration and/or movement and/or orientation changes. In some situations, a malfunction of a mechanical subsystem may cause the entire MEMS sensor to go into an error mode of operation, where it, as observed over its electronic interface, clearly is not operating according to specifications. Here, the knowledge of a sensor malfunction may then be interpreted by an onboard system controller and suitable risk mitigation can be executed, for example to sound an alarm to the user and discontinue further use of the product.

In other situations, and/or for other types of sensors not including MEMS sensors, potentially the malfunction within the mechanical sensor subsystem will not be obvious from reading its electronic interface. Only one or more of the individual acceleration signals might be completely irresponsive to external acceleration/movement/orientation. In this situation, any external detection might only be possible through performing a dedicated test in which the orientation sensor is systematically exposed to certain known acceleration, movement and/or orientation, where subsequently this controlled exposure is compared with the signals read from the orientation sensor.

If the infusion device 100 experiences physical impacts, for example during a drop to the floor and/or bumping into something, the orientation sensor 114 may register a resulting acceleration pattern in the multiple of spatial orientations. A suitable software algorithm may potentially be able to analyse, and hence potentially be able to detect the severity of such an impact, e.g. in terms of the total acceleration magnitude, and interpret possible causes to certain acceleration profiles plus determining the risk of whether or not the infusion device 100 might have suffered any critical damage. For example, during a drop to the floor, an integrated accelerometer will typically register a pattern of normal gravitation of 1 g downwards orientated, changing to zero (or close to) acceleration during the free-fall, then a very high acceleration peak (hitting the floor) ultimately followed by stable normal 1 g gravitational acceleration afterwards. If observing this acceleration pattern, then possibly the infusion device 100 must be deemed inadequate for further use and appropriate audible and visual alarms may be issued to the user. Certainly, if the drop is detected but the orientation/acceleration signal doesn't not come on after the impact, this indicates that the orientation sensor 114 might have been damaged during the episode, in which case the infusion device 100 is defective.

For bumping into an object, possibly this would result in a stable 1 g acceleration, changing to a peak acceleration, e.g. a significant acceleration peak magnitude and followingly changing back to a stable 1 g acceleration. Other cases may be developed to detect, and mitigate a risk of damaged device, based on typical user ‘misuse’ but in addition, such a detection sensor might also indicate other scenarios and/or patient conditions, for example it could detect patient posture and physical activity for a pump belt-worn by the patient, detect body tremors and also falling of the patient etc.

An orientation sensor self-test system 115 configured to test if the orientation sensor 114 is fully functional may therefore be included in the infusion device 100.

The main controller 112 may be configured to control functions of the infusion device 100 and to read the orientation signals from the orientation sensor 114 when the orientation of the infusion device 100 is relevant for performing such functions. The test of the orientation sensor 114 may include checking if the orientation sensor 114 outputs the orientation signals as it is supposed to do. Thus, if the test performed by the orientation sensor self-test system 115 shows that the orientation sensor 114 is different from a predefined value, e.g. within a pre-defined range, the orientation sensor self-test system 115 may sound an alarm. Alternatively, or in combination, a user of the infusion device 100 may be notified if the test of the orientation sensor 114 reveals a malfunction of the same, e.g. by a notification on the user interface 112 and/or audible noise generator, a tactile feedback device such as a vibratory device, or equivalent device providing auditory or tactile feedback for a user. The alarm may in one or more examples still be sounded and/or the user of the infusion device 100 otherwise notified about the malfunction of the orientation sensor 114 even if the malfunction is so severe that the orientation sensor 114, and potentially also the main controller 112 of the infusion device 100, shuts down and discontinues its operation.

The test of the orientation sensor 114 may include exposing the infusion device 100, and thereby the orientation sensor 114, to certain carefully controlled agitation stimuli, such as movement, acceleration and/or change of orientation, and checking whether the orientation signals output from the orientation sensor 114 correspond to the agitation stimuli, to which the orientation sensor 114 has been exposed. Thus, the orientation sensor self-test system 115 of the infusion device 100 may compare an expected orientation signals output expected to be obtained from the orientation sensor 114 based on the stimuli provided thereto with the actual orientation signals output from the orientation sensor 114. If the difference between the measured output signal and the expected output signal is too large, a malfunction is detected.

Alternatively, or in addition, the test of the orientation sensor 114 may include artificially skewing a gravitational field around the orientation sensor 114 thereby creating a testing reference stimulus, and checking whether the orientation signals output from the orientation sensor 114 correspond to the agitation stimuli, to which the orientation sensor 114 has been exposed. Again, if the difference between the measured output signal and the expected output signal is too large, a malfunction is detected. The skewing of the gravitational field around the orientation sensor 114 thereby creating the testing reference stimulus may be done without changing the physical orientation of the infusion device 100 and/or the orientation sensor 114.

A method of testing the function of the orientation sensor 114 may be to perform five tests in a normal mode and five tests in a self-test mode. If the difference between the highest and lowest test result is within a pre-determined range, the orientation sensor 114 may be seen as functioning as it is supposed to.

To better balance the risk of having a malfunctioning orientation sensor 114 with a reasonably acceptable level of user/operator involvement, taking into consideration that the orientation sensor 114 potentially could have been damaged at any time during the use-process, a systematic calibration every 1-2 weeks may not be sufficient to exercise for mitigations. Normally, the test of the orientation sensor 114 is therefore performed before reading the orientation signals by the main controller 112. Thus, the orientation sensor self-test system 115 may assess whether the orientation sensor 114 is functional or not before the user performs a further operations using the infusion device 100 and/or initiate medicament delivery. The test of the orientation sensor 114 may even be performed before every reading of the orientation signals by the main controller 112. Alternatively, the test of the orientation sensor 114 may even be performed before every second, third, fourth, fifth, sixth, seventh, eighth, ninth, tenth, or more reading of the orientation signals by the main controller 112. The test of the orientation sensor 114 may also and/or further be performed at every power on or off of the infusion device 100 and/or at regular intervals when the infusion device 100 is powered on, such as 1, 2 or 10 times during a day.

The orientation sensor 114 may be configured to monitor a multiple of spatial orientations of the infusion device 100 and to continuously output orientation signals containing information about the multiple of spatial orientations of the infusion device 100. For each of the multiple of spatial orientations, the test of the orientation sensor 114 may include repeated readings, such as 5-10 readings, of orientation signals corresponding to that specific dimension. For each of these orientation signals, a check for variation between the multiple readings of that specific orientation signal may be performed as a test of whether the orientation sensor 114 outputs the orientation signals as it is supposed to do.

The check for variation may include comparing the variations of the multiple readings to a predefined threshold value for each orientation signal so that the multiple readings of a given orientation signal are only deemed to vary if the variation exceeds the threshold value defined for that specific orientation signal.

An approach to continuously measuring a multiple of spatial orientations of the infusion device 100 using the orientation sensor 114 is to test whether the sensor orientation sensor 114 outputs ‘live’ signals at the time of needing to assess and/or control the infusion device orientation. While the generation of live signals will not guarantee the full 100% functional integrity of the orientation sensor 114, it may help to rule out any obvious malfunction scenarios related for example to a damaged mechanical MEMS sub-system no longer capable of moving and/or vibrating in response to e.g. an external acceleration. Thus, if live signals continuously are generated from the orientation sensor 114, this may indicate a functional orientation sensor 114.

Practically, the test for live signals from the orientation sensor 114 may be carried out by executing a sequence of repeated readings of the orientation sensor 114, for example 5-10 repetitions, and analyzing these to ensure, that the individual readings are all different. The readings may be obtained for each spatial orientation, e.g. three axes in a X, Y, Z sensor, in parallel because damage could potentially have occurred for individual spatial orientation, e.g. only one out of three axes thus leading to potentially observing stable data readings for those individual sensor axes should any defect be present in those affected portions of the orientation sensor 114.

Since the reading of the orientation sensor 114 is usually taking place at fairly high rates, for example one complete reading could easily be performed 100-1000 times per second (or higher), the practical burden and time-delay associated with checking for ‘live’ sensor signals may easily be incorporated at every single point in a use process where the sensor data is to be measured. If reading for example 10 data values subsequent of each other at a frequency of 100 Hz or 1000 Hz only takes 0.01-0.1 seconds, this reading will normally not be a limiting component in any use process step. Thus, ‘live’ check may be added in front of any attempt to read data from the orientation sensor 114 and an alarm may optionally be sounded if the data are stable for this ‘live’ check assessment indicating a malfunctioning sensor.

Care may possibly need to be taken to integrate the ‘live’ check with the actual use pattern, and as such ensure to set appropriate thresholds for the comparison of change between individual readings, which may in fact be larger than the least significant bit/digit on the sensor readout. While the assumption will be that the readout data is stable down to the least possible change, and that some marginal vibration will be assumed even for a device with the sensor resting on a tabletop, a slightly elevated threshold of required variation between readout samples could strengthen the validity of the check cycle (although possibly also enhance the chance of false-positives).

In often seen applications, the orientation signal may be a precondition, e.g. the control system may be provided with precondition settings, such that the user may only activate, e.g. a priming activation button following registration of the orientation being acceptable by the orientation sensor self-test system 115 testing if the orientation sensor 114 is fully functional. Alternatively, the priming activation can be automated (within a programmed sequence and/or based on an earlier user button activation) so that the priming then initiates when the orientation becomes acceptable.

During e.g. a priming process, the injection device 100 may display instructions to the user on the user interface 102. These instructions are often key to the user's ease of use. However, when handling the injector device 100 and getting the orientation of the same adjusted for optimum priming having any air-bubbles on the part of the syringe compartment 204 pointing upwards, it may conflict with the text and/or icons shown on the on-screen user interface 102. This may make it difficult to keep reading these instructions. Therefore, the orientation sensor 114 may also be configured to feed orientation information to a graphical display controller so that the display text and/or icons can re-arrange/rotate to the ‘optimal’ orientation based on the device's physical orientation (for example invert when the user inverts the device orientation). Thus, the infusion device 100 may further comprise a graphical display, e.g. the user interface 102, and the main controller 112 may be configured to use the orientation signals from the orientation sensor 114 to rotate information, such as text and icons, on the graphical display depending on the orientation of the infusion device 100. By rotating the information, it is possible to ensure that the information is always facing in an optimal orientation for facilitating the reading thereof by a user of the infusion device 100. This feature may be a huge help to ensure readability of the displayed instructions, but may to some users be an annoyance if they are very familiar with the usage of the infusion device 100 without this feature. Therefore, an activation/deactivation based on the user's preference settings may be included in the infusion device 100.

When the plunger interface is implemented for both pushing and pulling, where the lead screw 108 is configured to move the plunger 202 in the first direction and the second direction, opposite the first direction, the syringe compartment 204 may be filled with medicament from an attached vial. Thus, an empty syringe 200 installed in the infusion device 100, may be filled as part of preparing for a subsequent infusion using the same infusion device 100 for both filling and infusion. Thus, the main controller 112 may be configured to use orientation signals from the orientation sensor 114 for facilitating a correct filling of the syringe 200 from a vial attached to the infusion device 100 by ensuring that the plunger 202 can only be moved in the direction out of the syringe 200, thereby withdrawing a fluid from the vial into the syringe 200, when the infusion device 100 is oriented so that the syringe 200 is in an approximately vertical position with the vial on top.

This is useful in a therapy where a liquid drug product is only provided in a simple vial and not in a syringe suitable for being received in the infusion device 100. An empty syringe 200 may be installed inside the infusion device 100 and at its front, a standard Luer-connector vial adaptor may be attached. The standard Luer-connector vial adaptor may on its other end be connected to the vial through a spike or a needle penetrating a silicone septum closure of the vial. The infusion device 100 may therefore be used following a ‘normal’ practice from filling a fluid into a syringe from a vial, including for example injection of air from the syringe into the vial before withdrawing a similar amount of volume liquid drug from the vial. In such situation, the use of the orientation sensor, e.g. an X, Y, Z accelerometer will again be highly valuable in making sure that the liquid drug (and not just air) is pulled over to the syringe. The orientation sensor may determine if the vial is above the syringe, hence if the liquid inside the vial will flow to the vial adaptor spike/needle, and as such control the infusion device electronics and motor etc. to only allow plunger rod withdrawal when the system is approximately vertical with the vial on top. The system may pull-back the plunger rod to transfer all liquid from the external vial (continue until end-stop for the plunger rod travel) or alternatively leverage the fine-accuracy rotational sensor of the motor to displace the plunger rod a precise pre-set volume distance, thus aspirating a precise pre-set amount of liquid from the vial. After having transferred the liquid drug substance from the vial into the syringe, the vial adaptor (with the vial) may easily be unscrewed at the Luer connection, and a suitable infusion line may be attached for example for subcutaneous or intravenous infusion. Before inserting the needle and executing the infusion, priming will likely be required again leveraging the orientation sensor (as described above).

In another example, an external vial may contain the powder/cake from a lyophilized drug which is to be reconstituted into a liquid substance before infusion. With the lyophilized drug provided in the vial, a syringe to be installed inside the infusion device may be prefilled with an accompanying diluent. The two primary containers may be connected using a standard vial adaptor like described above. With the combined system attached to the infusion device, the infusion device may now execute a pre-programmed sequence of injecting the diluent into the vial, and instruct the user to swirl the vial to facilitate complete reconstitution of the drug product. Then, after having verified a perfectly reconstituted drug product is obtained, the infusion device may be used for assisting to fill the liquid drug product back into the syringe following the same approach as the above-described filling of a syringe from an external vial using the infusion device. The orientation sensor may potentially be vital in securing optimal orientation of the infusion device at the various stages of the process which, beyond the final withdrawal back into the syringe, also easily may include aspects of ensuring an appropriate orientation of the infusion device during initial injection of diluent into the lyophilized powder, e.g. to minimize foaming creation. Having the vial on top compared to the syringe position may be preferable to ensure that a jet of diluent does not inject forcefully into a lyophilized cake/powder thereby causing foaming, e.g. through separation of the cake/powder substance that would enable air to get trapped between individual particles here.

During the swirling reconstitution process, the orientation sensor may possibly be of high value in monitoring the swirling movements induced by the user/operator. When instructed to swirl in such situations, typically the intention is to apply a gentle rotating motion to the vial containing the two mixed drug components, appreciating that possibly a too forceful ‘shaking’ might risk trapping air in the mixed substance, hence possible resulting in foaming which might ultimately carry risk in terms of an unprecise dose and/or injecting air into the patient (e.g. SC tissue, vein or muscle etc.). With the signal from the orientation sensor, the infusion device may be able to monitor and sound an alarm should the user shake too forcefully when he is instructed to only apply a gentle swirling.

Thus, the main controller 112 may be configured to use orientation signals from the orientation sensor 114 for facilitating a pre-programmed sequence of injecting a diluent into a vial containing a powder of a lyophilised drug, diluting the powder within the vial to obtain a reconstituted drug solution, and withdrawing the liquefied drug from the vial into the syringe 200 by ensuring that the vial is in the right position relative to the syringe 200, such as below the syringe 200 when the diluent is injected into the vial and on top of the syringe 200 when the reconstituted drug solution is withdrawn into the syringe 200.

The main controller 112 may further be configured to use the orientation signals from the orientation sensor 114 for monitoring a manual swirling of the vial performed by a user of the infusion device 100 for diluting the powder therein and to inform the user, such as by sounding an alarm, if the vial is shaken too forcefully and/or when the swirling is considered to be sufficient and the drug is expected to be fully reconstituted.

If the requirement would be to ensure a vigorous swirling in order to mix the drug components, i.e. during the reconstitution process the user were to apply a forceful shaking and/or number of rotational inversions etc., the orientation sensor may be able to monitor the actual movements (orientation and magnitude of the induced acceleration) and sound an alarm if the handling is evaluated to be too weak for the desired effect.

For the above-described types of monitoring, a 3-axis gyroscope and/or one or more 3-axis accelerometer(s) may be used.

In addition to leverage of an acceleration signal, the infusion device may also be able to incorporate parameters of overall timing duration so that a certain level of acceleration has to be present for at least a certain period of time. Adding further to this, other parameters might influence the resulting reconstitution quality, such as for example environmental temperature etc.

Compared to typical scenarios for lyophilized drug products needing dissolving reconstitution, some drugs will not dissolve into a liquid but rather transform into a suspension which then may support infusion into the human tissue. The requirements to a suspension for supporting injected administration may typically be on its level of homogeneity. However, the ability of a certain suspension to stay homogenous over time depends on many parameters. This means, following the examples from above, that apart from all preparation steps performed, also the time from withdrawal into the syringe and until the drug is actually infused might become a separate risk to be additionally confined. A timer inside the infusion device may be able to monitor the pauses between the various process steps and sound an alert should the user attempt to infuse a reconstituted suspension after a too long waiting time. This could trigger an instruction to either shake the syringe with the drug, while being monitored using the orientation sensor, before being accepted to proceed to infusion.

Some suspensions, however, are so viscous that they will not easily be physically agitated sitting inside the syringe with no air. Hence, re-suspension could dictate remounting of the vial to the syringe Luer connection, using the vial adaptor, injecting the suspension into the vial, carrying through the appropriate re-suspension by physically shaking the vial, then filling the re-suspended drug back into the syringe, dismounting the vial, priming the syringe and infusion line and then carry through the infusion.

The infusion device 100 may be used in a method for testing an orientation sensor 114 of an infusion device 100. The orientation sensor 114 may be configured to monitor a spatial orientation of the infusion device 100 and to continuously output orientation signals containing information about the orientation of the infusion device 100. The infusion device 100 may further comprise a main controller 112 configured to control functions of the infusion device 100 and to read the orientation signals from the orientation sensor 114 when the orientation of the infusion device 100 is relevant for performing such functions. The method may comprise the step of checking if the orientation sensor 114 outputs the orientation signals as it is supposed to do, wherein the step of checking if the orientation sensor 114 outputs the orientation signals as it is supposed to do is performed before reading the orientation signals by the main controller 112.

The method may comprise a step of sounding an alarm and/or otherwise notifying a user of the infusion device 100 if a malfunction of the orientation sensor 114 is detected.

The step of sounding the alarm and/or otherwise notifying the user of the infusion device 100 may be performed even if the malfunction of the orientation sensor 114 is so severe that the orientation sensor 114, and potentially also the main controller 112 of the infusion device 100, shuts down and discontinues its operation.

The step of checking if the orientation sensor 114 outputs the orientation signals as it is supposed to do may be performed before every reading of the orientation signals by the main controller 112.

The method may further comprise the steps of reading repeatedly, such as 5 times or 10 times, for each spatial dimension, orientation signals corresponding to that specific dimension, and checking, for each of these orientation signals, for variation between the multiple readings of that specific orientation signal.

The step of checking for variation includes a predefined threshold value for each orientation signal so that the multiple readings of a given orientation signal may only be deemed to vary if the variation exceeds the threshold value defined for that specific orientation signal.

The steps of the method may be performed at every power on or off of the infusion device 100 and/or at regular intervals when the infusion device 100 is powered on, such as 1, 2 or 10 times during a day.

The method may further comprise the steps of exposing the infusion device 100, and thereby the orientation sensor 114, to certain carefully controlled agitation stimuli, such as movement, acceleration and/or change of orientation, and checking whether the orientation signals output from the orientation sensor 114 correspond to the agitation stimuli, to which the orientation sensor 114 has been exposed.

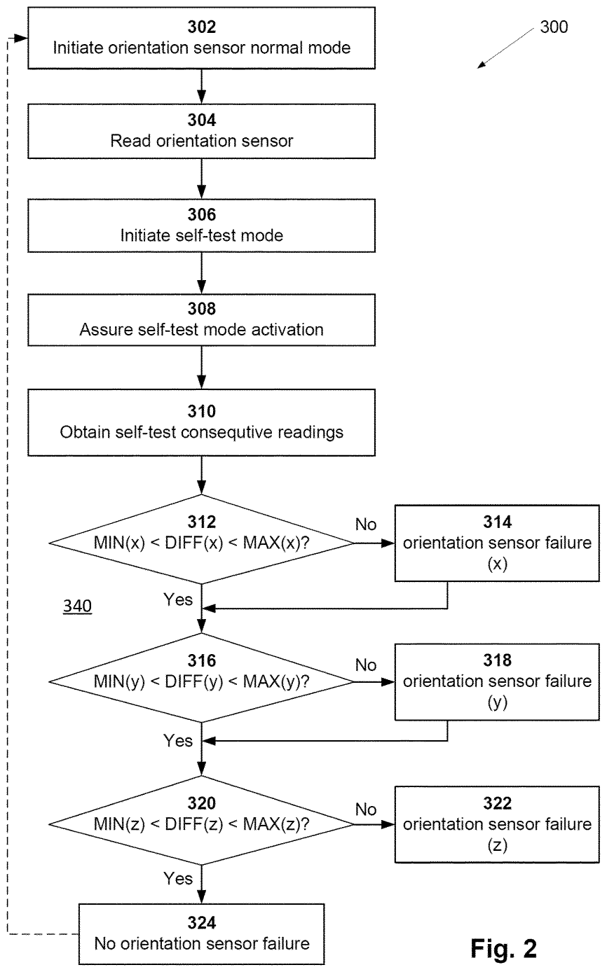

FIG. 2 shows an example of a method of testing the function of the orientation sensor 114. In a first step 302, the orientation sensor 114 is initiated. After initiation of the orientation sensor 114, orientation sensor signal(s) are read in the second step 304. Thereafter, a self-test mode of the orientation sensor is initiated in step 306. Before obtaining readings in the self-test mode, a short waiting period of e.g. 100 milliseconds is performed in step 308. After obtaining a number of consecutive readings, e.g. 5 readings, in the self-test mode in step 310, each reading being performed at a predetermined time interval of e.g. 20 milliseconds, the test results are evaluated in steps 312, 316, and 320.

Normally, the evaluation is performed for each spatial orientation. FIG. 2 shows an example of an evaluation in three spatial orientations, e.g. in the X, Y, Z orientation.

If the difference, DIFF(x), between the normal reading in step 304 in a first spatial orientation and the readings in the self-test mode in step 310 in the first spatial orientation is either below a pre-determined min value, MIN(x), for the first spatial orientation or above a pre-determined max value, MAX(x), for the first spatial orientation, i.e. if the difference is outside a pre-determined interval for the first spatial orientation defined by the pre-determined min value and the pre-determined max value for the first spatial orientation, an orientation sensor failure has been detected in the first spatial orientation and an output to the user will be provided in step 314. If the difference DIFF(x) is inside the pre-determined interval defined by the pre-determined min value and the pre-determined max value, no orientation sensor failure will be detected for the first spatial orientation.

If the difference, DIFF(y), between the normal reading in step 304 in a second spatial orientation and the readings in the self-test mode in step 310 in the second spatial orientation is either below a pre-determined min value, MIN(y), for the second spatial orientation or above a pre-determined max value, MAX(y), for the second spatial orientation, i.e. if the difference is outside a pre-determined interval for the second spatial orientation defined by the pre-determined min value and the pre-determined max value for the second spatial orientation, an orientation sensor failure has been detected in the second spatial orientation and an output to the user will be provided in step 318. If the difference DIFF(y) is inside the pre-determined interval defined by the pre-determined min value and the pre-determined max value, no orientation sensor failure will be detected for the second spatial orientation.

If the difference, DIFF(z), between the normal reading in step 304 in a third spatial orientation and the readings in the self-test mode in step 310 in the third spatial orientation is either below a pre-determined min value, MIN(z), for the third spatial orientation or above a pre-determined max value, MAX(z), for the third spatial orientation, i.e. if the difference is outside a pre-determined interval for the third spatial orientation defined by the pre-determined min value and the pre-determined max value for the third spatial orientation, an orientation sensor failure has been detected in the third spatial orientation and an output to the user will be provided in step 322. If the difference DIFF(z) is inside the pre-determined interval defined by the pre-determined min value and the pre-determined max value, no orientation sensor failure will be detected for the third spatial orientation.

If no orientation sensor failure is detected in any of the spatial orientations, the orientation sensor 114 is working as it is supposed to. This may possibly be indication to the user in step 324. If no orientation sensor failure is detected, the infusion device is ready for a new self-test starting from step 302.

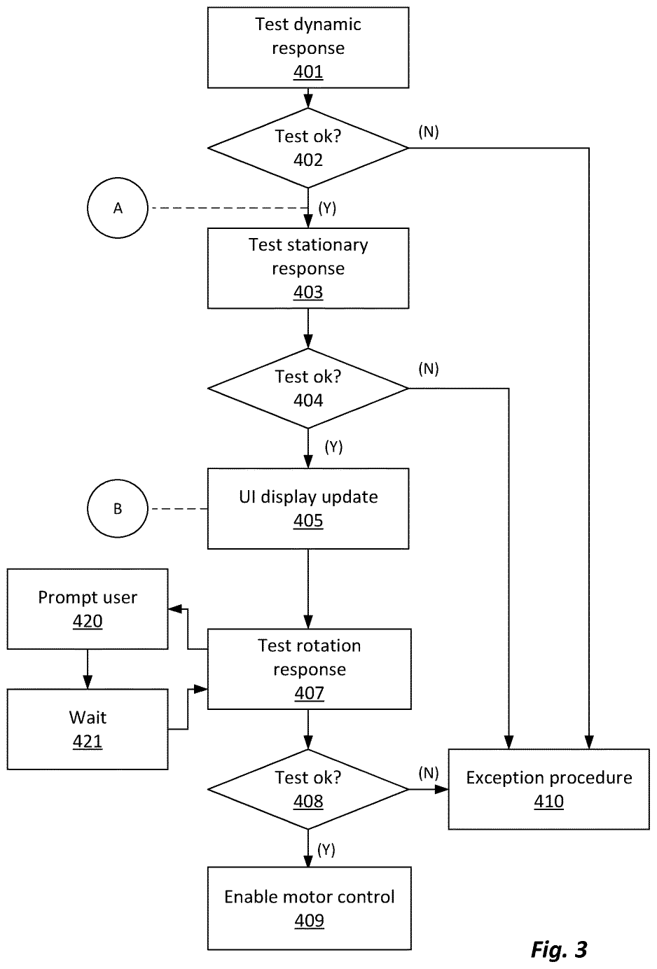

FIG. 3 shows a flowchart for a method at the infusion device. The method includes several determinations as conditions for enabling the main controller to operatively control the motor. As mentioned herein, the orientation sensor 114, includes an accelerometer with a suspended mass, configured to output a first signal including acceleration information and orientation information.

In step 401 the orientation sensor is tested in terms of a dynamic response corresponding with a movement, e.g. a slight shaking of the infusion device. According to a first determination 402, at a first time, a variability of magnitudes of the first signal fulfils (Y) a first criterion; wherein the first criterion includes a first threshold. Thus, if the movement causes a sufficient variability of magnitudes, it is deemed likely that the mass suspended in the orientation sensor is responsive to movement as intended. In the alternative, that the first criterion is not fulfilled (N), the method proceeds according to an exception procedure 410. The test of the dynamic response corresponds with steps 306, 308 and 310 in FIG. 2.

In the below, please note that the third determination, if performed, relates to a determination that is performed before the second determination. In some examples, the third determination is dispensed with or is an optional determination.

Subsequently, in step 403 the orientation sensor is tested in terms of a static response corresponding with a predefined orientation, e.g. corresponding with the infusion device pointing downwards. According to a third determination 404, at a third time, magnitudes of the first signal fulfil (Y) a third criterion; wherein the third criterion corresponds with a second range of orientations of the infusion device e.g. corresponding with the infusion device pointing downwards. In the alternative, that the third criterion is not fulfilled (N), the method proceeds according to the exception procedure 410. The test of the static response corresponds with steps 312 through 324 in FIG. 2.

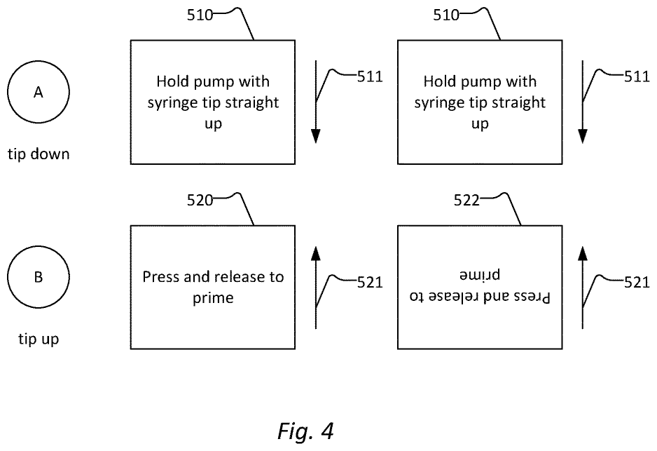

In some examples, the user interface 102 or a display included in the user interface is updated in step 405. This may include displaying a second display image (in accordance with a second display orientation) to replace a previous displayed, first display image (in accordance with a first display orientation) or rotating a display image to account for the infusion device being turned about a horizontal axis. See also FIG. 4. In some examples the first display orientation and the second display orientation differs by 180 degrees. For example, at the point ‘A’ in the flowchart, the first display image is shown in accordance with the first display orientation. For example, at the point ‘B’ in the flowchart, the second display image is shown in accordance with the second display orientation.

Subsequently, in step 407 it is tested whether the infusion device is currently handled to assume a correct orientation, e.g. corresponding with the infusion device pointing upwards, and was turned relative to the orientation the infusion device had during the static response test in step 403. According to a second determination 408, at a second time, a mean or median of magnitudes of the first signal fulfil (Y) a second criterion; wherein the second criterion corresponds with the first range of orientations of the infusion device e.g. corresponding with the infusion device pointing upwards. In the alternative, that the second criterion is not fulfilled (N), the method proceeds according to the exception procedure 410.

If each of the determinations were successful, the main controller 112 is enabled in step 409 to operatively control the motor 106 and movement of the plunger driver at a first period of time.

In some examples, test of the rotation response includes prompting a user at step 420 via an output unit, such as a display, a sound generator, and/or a haptic generator, to guide a user to change orientation of the infusion device. In subsequent step 421 the method waits for a predetermined period of time to give the user sufficient time to turn the infusion device.

FIG. 4 shows examples of display images and display updates. At point A of the flowchart in FIG. 3, the infusion device has, if correctly handled, an orientation corresponding to the syringe opening 101 and the syringe outlet 208 pointing downwards (as illustrated by arrow 511). A first display image 510 may accordingly include instructions “Hold pump with syringe tip straight up” to instruct the user to turn the infusion device 180 degrees about the horizontal axis. The displaying of the first display image may depend on a current orientation of the infusion device, one ore more preceding user interactions, and/or a successful first determination.

At point B of the flowchart in FIG. 3, the infusion device has, if correctly handled in response to the instruction in the first display image 510, an orientation corresponding to the syringe opening 101 and the syringe outlet 208 pointing upwards (as illustrated by arrow 521). A second display image 520 may accordingly include instructions “Press and release to prime” to instruct the user to activate a priming action. The displaying of the second display image may depend on the current orientation of the infusion device, one ore more preceding user interactions, and/or a successful third determination e.g. including a successful second determination.

As illustrated, the first display image 510 is shown in accordance with a first display orientation and the second display image 520 is shown in accordance with a second display orientation to account for the infusion device being turned. The second display orientation corresponds to a 180 degrees rotation relative to the first display orientation.

As illustrated with respect to the second display image 522, had the display orientation not been changed, the display image would be difficult to read when the device is turned.

Generally, the above improves the safety associated with a user's use of the infusion device.

### Items

The invention is further described in the following items.
1. An infusion device (100) comprising:
   a receptacle (120) configured to receive a syringe (200) comprising a plunger (202);
   a plunger driver (104) configured to releasably engage an end of the plunger (202) of the syringe (200) and to move the plunger (202) in a longitudinal direction of the syringe (200);
   a drive train (105) comprising:
      a rotating motor (106), such as a DC motor;
      a lead screw (108) onto which a plunger driver (104) is mounted; and
      one or more gears (110) configured to couple a shaft of the motor (106) to the lead screw (108);
   an orientation sensor (114) configured to monitor a spatial orientation of the infusion device (100) and to continuously output orientation signals containing information about the spatial orientation of the infusion device (100);
   an orientation sensor self-test system (115) configured to test if the orientation sensor (114) is fully functional; and
   a main controller (112) configured to control functions of the infusion device (100) and to read the orientation signals from the orientation sensor (114) when the orientation of the infusion device (100) is relevant for performing such functions,
wherein the test of the orientation sensor (114) includes checking if the orientation sensor (114) outputs the orientation signals as it is supposed to do, and
wherein the test of the orientation sensor (114) is performed before reading the orientation signals by the main controller (112).
2. The infusion device (100) according to item 1, wherein an alarm is sounded and/or a user of the infusion device (100) is otherwise notified if the test of the orientation sensor (114) reveals a malfunction of the same.
3. The infusion device (100) according to item Error! Reference source not found., wherein the alarm is sounded and/or the user of the infusion device (100) is otherwise notified about the malfunction of the orientation sensor (114) even if the malfunction is so severe that the orientation sensor (114), and potentially also the main controller (112) of the infusion device (100), shuts down and discontinues its operation.
4. The infusion device (100) according to any preceding item, wherein the test of the orientation sensor (114) is performed before every reading of the orientation signals by the main controller (112).
5. The infusion device (100) according to any preceding item, wherein spatial orientation is an orientation in three dimensions, such as an orientation in an X, Y, Z dimension.
6. The infusion device (100) according to any preceding item, wherein the orientation sensor (114) is configured to monitor a multiple of spatial orientations of the infusion device (100) and to continuously output orientation signals containing information about the multiple of spatial orientations of the infusion device (100), wherein, for each of the multiple of spatial orientations, the test of the orientation sensor (114) includes repeated readings, such as 5-10 readings, of orientation signals corresponding to that specific dimension and, for each of these orientation signals, a check for variation between the multiple readings of that specific orientation signal.
7. The infusion device (100) according to item 6, wherein the check for variation includes a predefined threshold value for each orientation signal so that the multiple readings of a given orientation signal are only deemed to vary if the variation exceeds the threshold value defined for that specific orientation signal.
8. The infusion device (100) according to any preceding item, wherein the test of the orientation sensor (114) is further performed at every power on or off of the infusion device (100) and/or at regular intervals when the infusion device (100) is powered on, such as 1, 2 or 10 times during a day.
9. The infusion device (100) according to any preceding item, further comprising a graphical display,
   wherein the main controller (112) is configured to use the orientation signals from the orientation sensor (114) to rotate information, such as text and icons, on the graphical display depending on the orientation of the infusion device (100).
10. The infusion device (100) according to any preceding item, wherein the test of the orientation sensor (114) includes:
   exposing the infusion device (100), and thereby the orientation sensor (114), to certain carefully controlled agitation stimuli, such as movement, acceleration and/or change of orientation; and
   checking whether the orientation signals output from the orientation sensor (114) correspond to the agitation stimuli, to which the orientation sensor (114) has been exposed.
11. The infusion device (100) according to any preceding item, wherein the test of the orientation sensor (114) includes:
   artificially skewing a gravitational field around the orientation sensor (114) thereby creating a testing reference stimulus; and
   checking whether the orientation signals output from the orientation sensor (114) correspond to the agitation stimuli, to which the orientation sensor (114) has been exposed.
12. The infusion device (100) according to any preceding item, wherein the orientation sensor (114) is a micro electro-mechanical system (MEMS), such as a 3-axes accelerometer, or a magnetometer, such as a 3-axes magnetometer.
13. The infusion device (100) according to any preceding item, wherein the plunger driver (104) is configured to engage the plunger (202) of the syringe (200) in such a way that the drive train (105) of the infusion device (100) is able to move the plunger (202) in both directions, i.e. in the direction out of the syringe (200) as well as in the direction into the syringe (200).
14. The infusion device (100) according to item 13, wherein the main controller (112) is configured to use orientation signals from the orientation sensor (114) for:
   facilitating a correct filling of the syringe (200) from a vial attached to the infusion device (100) by ensuring that the plunger (202) can only be moved in the direction out of the syringe (200), thereby withdrawing a fluid from the vial into the syringe (200), when the infusion device (100) is oriented so that the syringe (200) is in an approximately vertical position with the vial on top.
15. The infusion device (100) according to item 13 or 14, wherein the main controller (112) is configured to use orientation signals from the orientation sensor (114) for:
   facilitating a pre-programmed sequence of injecting a diluent into a vial containing a powder of a lyophilised drug;
   awaiting the powder within the vial to obtain a reconstituted drug solution; and
   withdrawing the liquefied drug from the vial into the syringe (200) by ensuring that the vial is in the right position relative to the syringe (200), such as below the syringe (200) when the diluent is injected into the vial and on top of the syringe (200) when the reconstituted drug solution is withdrawn into the syringe (200).
16. The infusion device (100) according to item 15, wherein the main controller (112) is further configured to use the orientation signals from the orientation sensor (114) for monitoring a manual swirling of the vial performed by a user of the infusion device (100) for diluting the powder therein and to inform the user, such as by sounding an alarm, if the vial is shaken to forcefully and/or when the swirling is considered to be sufficient and the drug is expected to be fully reconstituted.
17. A method for testing an orientation sensor (114) of an infusion device (100), the orientation sensor (114) being configured to monitor a spatial orientation of the infusion device (100) and to continuously output orientation signals containing information about the orientation of the infusion device (100),
   wherein the infusion device (100) further comprises a main controller (112) configured to control functions of the infusion device (100) and to read the orientation signals from the orientation sensor (114) when the orientation of the infusion device (100) is relevant for performing such functions,
   wherein the method comprises the step of checking if the orientation sensor (114) outputs the orientation signals as it is supposed to do, and
   wherein the step of checking if the orientation sensor (114) outputs the orientation signals as it is supposed to do is performed before reading the orientation signals by the main controller (112).
18. The method according to item 17, further comprising the step of sounding an alarm and/or otherwise notifying a user of the infusion device (100) if a malfunction of the orientation sensor (114) is detected.
19. The method according to item 18, wherein the step of sounding the alarm and/or otherwise notifying the user of the infusion device (100) is performed even if the malfunction of the orientation sensor (114) is so severe that the orientation sensor (114), and potentially also the main controller (112) of the infusion device (100), shuts down and discontinues its operation.
20. The method according to any of items 17-19, wherein the step of checking if the orientation sensor (114) outputs the orientation signals as it is supposed to do is performed before every reading of the orientation signals by the main controller (112).
21. The method according to any of items 17-20, wherein the method further comprises the steps of:
   reading repeatedly, such as 5 times or 10 times, for each spatial dimension, orientation signals corresponding to that specific dimension, and
   checking, for each of these orientation signals, for variation between the multiple readings of that specific orientation signal.
22. The method according to item 21, wherein the step of checking for variation includes a predefined threshold value for each orientation signal so that the multiple readings of a given orientation signal are only deemed to vary if the variation exceeds the threshold value defined for that specific orientation signal.
23. The method according to any of items 17-22, wherein the steps of the method are performed at every power on or off of the infusion device (100) and/or at regular intervals when the infusion device (100) is powered on, such as 1, 2 or 10 times during a day.
24. The method according to any of items 17-23, wherein the method further comprises the steps of:
   exposing the infusion device (100), and thereby the orientation sensor (114), to certain carefully controlled agitation stimuli, such as movement, acceleration and/or change of orientation, and
   checking whether the orientation signals output from the orientation sensor (114) correspond to the agitation stimuli, to which the orientation sensor (114) has been exposed.

### Summary of Items

Disclosed herein in a first aspect is an infusion device comprising:
a receptacle configured to receive a syringe comprising a plunger;
a plunger driver configured to releasably engage an end of the plunger of the syringe and to move the plunger in a longitudinal direction of the syringe;
a drive train comprising:
a rotating motor, such as a DC motor;
a lead screw onto which a plunger driver is mounted; and
one or more gears configured to couple a shaft of the motor to the lead screw;
an orientation sensor configured to monitor a spatial orientation of the infusion device and to continuously output orientation signals containing information about the spatial orientation of the infusion device;
an orientation sensor self-test system configured to test if the orientation sensor is fully functional; and
a main controller configured to control functions of the infusion device and to read the orientation signals from the orientation sensor when the orientation of the infusion device is relevant for performing such functions,
   wherein the test of the orientation sensor includes checking if the orientation sensor outputs the orientation signals as it is supposed to do, and
   wherein the test of the orientation sensor is performed before reading the orientation signals by the main controller.

By the orientation sensor outputting signals as it is supposed to do may be meant that it is expected to output values within a specific range. Thus, if a test performed by the orientation sensor self-test system shows that the orientation sensor is different from a predefined value, e.g. within a pre-defined range, or outside a pre-defined range of values, the orientation sensor self-test system may be seen as not functioning as it is supposed to.

By using an orientation sensor self-test system for testing the functioning of the orientation sensor before reading the orientation signals by the main controller is obtained a device, where it is always ensured that the orientation sensor is fully functional. Thus, situations where the orientation sensor gets stuck, and therefore outputs incorrect information, is avoided. This is for example relevant when performing a priming step, i.e. performing an air shot prior to medicament delivery to ensure that air has been evacuated from the syringe, since the priming step requires a correct orientation of the infusion device in order for air to evacuate the syringe. If the orientation sensor is not functioning as it is supposed to, medicament and not air may be evacuated from the infusion device. The air may subsequently be delivered to the patient instead of the correct medicament dose.

To balance the risk of a malfunctioning orientation sensor with a reasonably acceptable level of user/operator involvement, taking into consideration that the orientation sensor potentially could have been damaged at any time during a use-process, a calibration every 1-2 weeks will not be sufficient to exercise for mitigations. The use of the orientation sensor self-test system and the test of the orientation sensor being performed before reading the orientation signals by the main controller, ensures that the orientation sensor is fully functional at all times during operation and not just after a standard calibration, for example every 1-2 weeks.

In one or more examples, an alarm is sounded and/or a user of the infusion device is otherwise notified if the test of the orientation sensor reveals a malfunction of the same. In this manner, the user is able to react immediately if the orientation sensor is not outputting signals as it is supposed to.

In one or more examples, the alarm is sounded and/or the user of the infusion device is otherwise notified about the malfunction of the orientation sensor even if the malfunction is so severe that the orientation sensor, and potentially also the main controller of the infusion device, shuts down and discontinues its operation. This further ensures that the device does not shut down without notifying the user clearly of its malfunctioning.

In one or more examples, the test of the orientation sensor is performed before every reading of the orientation signals by the main controller. The test of the orientation sensor may be set for testing before every second reading or at a lower interval as well. However, by testing before every reading provides an optimal security that the orientation sensor is functioning as it is supposed to.

In one or more examples, spatial orientation is an orientation in three dimensions, such as an orientation in an X, Y, Z dimension. The orientation sensor may therefore e.g. by a three dimensions orientation sensor.

In one or more examples, the orientation sensor is configured to monitor a multiple of spatial orientations of the infusion device and to continuously output orientation signals containing information about the multiple of spatial orientations of the infusion device, wherein, for each of the multiple of spatial orientations, the test of the orientation sensor includes repeated readings, such as 5-10 readings, of orientation signals corresponding to that specific dimension and, for each of these orientation signals, a check for variation between the multiple readings of that specific orientation signal. The readings may thus be performed for each spatial orientation individually and information that the orientation sensor is not working as it is supposed to in one spatial orientation may be provided even though the orientation sensor is working as it is supposed to in other spatial orientations.

In one or more examples, the check for variation includes a predefined threshold value for each orientation signal so that the multiple readings of a given orientation signal are only deemed to vary if the variation exceeds the threshold value defined for that specific orientation signal. Thereby an incorrect reading on an otherwise well-functioning orientation sensor, may not influence the conclusion on whether the orientation sensor is working as it is supposed to or not.

In one or more examples, the test of the orientation sensor is further performed at every power on or off of the infusion device and/or at regular intervals when the infusion device is powered on, such as 1, 2 or 10 times during a day. The test frequency may thus be set individually for each infusion device.

In one or more examples, the infusion device further comprises a graphical display, wherein the main controller is configured to use the orientation signals from the orientation sensor to rotate information, such as text and icons, on the graphical display depending on the orientation of the infusion device. By rotating the information, it is possible to ensure that the information is always facing in an optimal orientation for facilitating the reading thereof by a user of the infusion device. This improves the reader experience and ensures that the relevant information is communicated in the most optimal manner as possible.

In one or more examples, the test of the orientation sensor includes exposing the infusion device, and thereby the orientation sensor, to certain carefully controlled agitation stimuli, such as movement, acceleration and/or change of orientation, and checking whether the orientation signals output from the orientation sensor correspond to the agitation stimuli, to which the orientation sensor has been exposed. In this manner, a reference value for what is expected is defined by the agitation stimuli provided to the device. A pre-set set of reference values may thus not be required, but instead defined according to the chosen agitation stimuli.

In one or more examples, the test of the orientation sensor includes artificially skewing a gravitational field around the orientation sensor thereby creating a testing reference stimulus, and checking whether the orientation signals output from the orientation sensor correspond to the agitation stimuli, to which the orientation sensor has been exposed. The artificial skewing of the gravitational field around the orientation sensor thereby creating the testing reference stimulus may be done without changing the physical orientation of the infusion device and/or the orientation sensor.

The orientation sensor may be a micro electro-mechanical system (MEMS), such as a 3-axes accelerometer, or a magnetometer, such as a 3-axes magnetometer. Other orientation sensor may alternatively also be imagined.

In one or more examples, the plunger driver is configured to engage the plunger of the syringe in such a way that the drive train of the infusion device is able to move the plunger in both directions, i.e. in the direction out of the syringe as well as in the direction into the syringe. The plunger driver may thus be used for delivery of medicament and for filling the syringe with medicament e.g. from a separate vial.

In one or more examples, the main controller is configured to use orientation signals from the orientation sensor for facilitating a correct filling of the syringe from a vial attached to the infusion device by ensuring that the plunger can only be moved in the direction out of the syringe, thereby withdrawing a fluid from the vial into the syringe, when the infusion device is oriented so that the syringe is in an approximately vertical position with the vial on top.

In one or more examples, the main controller is configured to use orientation signals from the orientation sensor for:
facilitating a pre-programmed sequence of injecting a diluent into a vial containing a powder of a lyophilised drug;
awaiting the powder within the vial to obtain a reconstituted drug solution; and
withdrawing the liquefied drug from the vial into the syringe by ensuring that the vial is in the right position relative to the syringe, such as below the syringe when the diluent is injected into the vial and on top of the syringe when the reconstituted drug solution is withdrawn into the syringe.

In one or more examples, the main controller is further configured to use the orientation signals from the orientation sensor for monitoring a manual swirling of the vial performed by a user of the infusion device for diluting the powder therein and to inform the user, such as by sounding an alarm, if the vial is shaken to forcefully and/or when the swirling is considered to be sufficient and the drug is expected to be fully reconstituted. This ensures an optimum preparation of the medicament/drug prior to delivery, which is relevant if a non-experienced user is preparing the medicament and the mixing procedure. A reference value used in this situation may be the acceleration in one or more spatial orientations evaluated individually or in combination. Further, derivatives of an acceleration signal, for example integration over time, thus providing an alternative average evaluation scheme that may better reflect user input over a period of time may also be used as reference.

Disclosed herein in a second aspect is a method for testing an orientation sensor of an infusion device, the orientation sensor being configured to monitor a spatial orientation of the infusion device and to continuously output orientation signals containing information about the orientation of the infusion device,

wherein the infusion device further comprises a main controller configured to control functions of the infusion device and to read the orientation signals from the orientation sensor when the orientation of the infusion device is relevant for performing such functions,
wherein the method comprises the step of checking if the orientation sensor outputs the orientation signals as it is supposed to do, and
wherein the step of checking if the orientation sensor outputs the orientation signals as it is supposed to do is performed before reading the orientation signals by the main controller.

By the orientation sensor outputting signals as it is supposed to do may be meant that it is expected to output values within a specific range. Thus, if a test performed by the orientation sensor self-test system shows that the orientation sensor is different from a predefined value, e.g. within a pre-defined range, or outside a pre-defined range of values, the orientation sensor self-test system may be seen as not functioning as it is supposed to.

By using an orientation sensor self-test system for testing the functioning of the orientation sensor before reading the orientation signals by the main controller is obtained a device, where it is always ensured that the orientation sensor is fully functional. Thus, situations where the orientation sensor gets stuck, and therefore outputs incorrect information, is avoided. This is for example relevant when performing a priming step, i.e. performing an air shot prior to medicament delivery to ensure that air has been evacuated from the syringe, since the priming step requires a correct orientation of the infusion device in order for air to evacuate the syringe. If the orientation sensor is not functioning as it is supposed to, medicament and not air may be evacuated from the infusion device. The air may subsequently be delivered to the patient instead of the correct medicament dose.

To balance the risk of a malfunctioning orientation sensor with a reasonably acceptable level of user/operator involvement, taking into consideration that the orientation sensor potentially could have been damaged at any time during a use-process, a calibration every 1-2 weeks will not be sufficient to exercise for mitigations. The use of the orientation sensor self-test system and the test of the orientation sensor being performed before reading the orientation signals by the main controller, ensures that the orientation sensor is fully functional at all times during operation and not just after a standard calibration every 1-2 weeks.

In one or more examples, the method further comprises the step of sounding an alarm and/or otherwise notifying a user of the infusion device if a malfunction of the orientation sensor is detected. In this manner, the user is able to react immediately if the orientation sensor is not outputting signals as it is supposed.

In one or more examples, the step of sounding the alarm and/or otherwise notifying the user of the infusion device is performed even if the malfunction of the orientation sensor is so severe that the orientation sensor, and potentially also the main controller of the infusion device, shuts down and discontinues its operation. This further ensures that the device does not shut down without notifying the user clearly of its malfunctioning.

In one or more examples, the step of checking if the orientation sensor outputs the orientation signals as it is supposed to do is performed before every reading of the orientation signals by the main controller. The test of the orientation sensor may be set for testing before every second reading or at a lower interval as well. However, by testing before every reading provides an optimal security that the orientation sensor is functioning as it is supposed to.

In one or more examples, the method further comprises the steps of reading repeatedly, such as 5 times or 10 times, for each spatial dimension, orientation signals corresponding to that specific dimension, and checking, for each of these orientation signals, for variation between the multiple readings of that specific orientation signal.

In one or more examples, the step of checking for variation includes a predefined threshold value for each orientation signal so that the multiple readings of a given orientation signal are only deemed to vary if the variation exceeds the threshold value defined for that specific orientation signal. Thereby an incorrect reading on an otherwise well-functioning orientation sensor, may not influence the conclusion on whether the orientation sensor is working as it is supposed or not.

In one or more examples, the steps of the method are performed at every power on or off of the infusion device and/or at regular intervals when the infusion device is powered on, such as 1, 2 or 10 times during a day. The test frequency may thus be set individually for each infusion device.

In one or more examples, the method further comprises the steps of exposing the infusion device, and thereby the orientation sensor, to certain carefully controlled agitation stimuli, such as movement, acceleration and/or change of orientation, and checking whether the orientation signals output from the orientation sensor correspond to the agitation stimuli, to which the orientation sensor has been exposed. In this manner, a reference value for what is expected is defined by the agitation stimuli provided to the device. A pre-set set of reference values may thus not be required, but instead defined according to the chosen agitation stimuli.

### REFERENCES

100 infusion device
101 syringe opening in the infusion device
102 user interface
103 plunger moving controllers
104 plunger driver
105 drive train
106 rotating motor
108 lead screw
110 gear
110a gear wheel
110b planetary gear
112 main controller
114 orientation sensor
115 orientation sensor self-test system
116 battery
117 driver
118 current measuring sensor
120 receptacle
122 housing
124 housing top part
126 housing bottom part
128 hinge
200 syringe
202 plunger
203 driver end of the plunger
204 medicament compartment
205 medicament end of the plunger
206 movable seal
208 syringe outlet
210 infusion line
212 needle
214 needle outlet
300 method for testing the functioning of the orientation sensor
302 initiate orientation sensor
304 read orientation sensor signal(s)
306 initiate self-test mode of the orientation sensor
308 wait to assure self-test mode activation
310 read a number of consecutive readings in the self-test mode with a pre-determined interval therein between
312 evaluation of the test results in a first spatial orientation
314 orientation sensor failure in the first spatial orientation
316 evaluation of the test results in a second spatial orientation
318 orientation sensor failure in the second spatial orientation
320 evaluation of the test results in a third spatial orientation
322 orientation sensor failure in the third spatial orientation
324 no orientation sensor failure
401 test dynamic response
402 test ok?
403 test stationary response
404 test ok?
405 UI display update
407 test rotation response
408 test ok?
409 enable motor control
410 exception procedure
420 prompt user
421 wait
510 first display image
520 second display image
522 second display image, at second display orientation
511 arrow, tip down
521 arrow, tip up

1. A method at an infusion device, wherein the infusion device includes:
a receptacle (120) configured to receive a syringe (200) comprising a plunger (202);
a plunger driver (104) configured to releasably engage an end of the plunger (202) of the syringe (200) and to move the plunger (202) in a longitudinal direction of the syringe (200);
a drivetrain (105), including a motor (106), coupled to move the plunger driver (104);
a main controller (112) configured to control travel of the plunger driver (104) in at least a first direction;
an orientation sensor (114), including an accelerometer with a suspended mass, configured to output a first signal including acceleration information and orientation information;
the method comprising:
a first determination (402), at a first time, that a variability of magnitudes of the first signal fulfils a first criterion; wherein the first criterion includes a first threshold;
a second determination (408), at a second time, that magnitudes of the first signal fulfil a second criterion; wherein the second criterion corresponds with a first range of orientations of the infusion device;
at least in accordance with the first determination and the second determination, enabling the main controller (112) to operatively control the motor (106) and movement of the plunger driver (104) at a first period of time.

2. A method according to claim 1, wherein the first signal output by the orientation sensor is associated with a noise floor level and a maximum magnitude; and wherein the first threshold is at a value above the noise floor level and below the maximum magnitude.

3. A method according to claim 1, comprising:
a third determination (404), at a third time, that magnitudes of the first signal fulfil a third criterion; wherein the third criterion corresponds with a second range of orientations of the infusion device; wherein the second range of orientations is different from the first range of orientations; wherein the third time is at or prior to the second time and at or after the first time; and
at least in accordance with the third determination, enabling the main controller (112) to operatively control movement of the motor (106) and movement of the plunger driver at the first period of time.

4. A method according to claim 1, comprising:
forgoing performing the second determination (407), in accordance with a determination that the second time does not fall within a first time period running from the third time.

5. A method according to claim 1, comprising:
in accordance with a failure to detect, using one or more detectors at the infusion device, at a time preceding the first time, that a user is handling the infusion device, forgoing performing the first determination (401).

6. A method according to claim 1, wherein one or both of the second determination (408) and the third determination (404) includes that a mean or median of magnitudes of the first signal fulfil the second criterion; wherein the mean or median of magnitudes of the first signal is determined over a period of time being shorter than about 500 milliseconds, e.g. shorter than about 300 milliseconds, e.g. about 100 milliseconds.

7. A method according to claim 1, comprising:
in accordance with a failure to determine that the first criterion is fulfilled at the first time and/or a failure to determine that the second criterion is fulfilled at the at the second time:
forgoing enabling the main controller (112) to operatively control the motor (106) and movement of the plunger driver at the first period of time; and
initiating a first exception procedure (410).

8. A method according to claim 3, comprising:
in accordance with a failure to determine that the third criterion is fulfilled at the third time:
forgoing enabling the main controller (112) to operatively control the motor (106) and movement of the plunger driver at the first period of time; and
initiating the first exception procedure (410).

9. A method according to claim 1, wherein the exception procedure includes:
in accordance with a failure to make the first determination (402), emitting a first message; wherein the first message prompts a user to restart the infusion device;
in accordance with a failure to make the second determination (408) and/or a failure to make the third determination (404), emitting a second message; wherein the second message guides a user to change orientation of the infusion device.

10. A method according to claim 1, wherein the infusion device is configured to receive a battery for supplying battery power to the main controller; the method comprising:
detecting a first event including a battery-power-on event; and
in response to detecting the first event, and a failure to make at least the first determination, initiating the first exception procedure.

11. A method according to claim 1, wherein the infusion device includes a first input element connected to the main controller for receiving a user input at the input element; the method comprising:
detecting a second event including a first input at the first input element;
in response to detecting the second event, and a failure to make at least the second determination (408) and/or a failure to make at least the third determination (404):
forgoing enabling the main controller (112) to operatively control the motor (106) and movement of the plunger driver at the first period of time; and
initiating the first exception procedure (410).

12. A method according to claim 1, wherein the infusion device includes a first output element connected to the controller; wherein the output unit includes one or more of: a display unit, a sound actuator, and a haptic actuator; the method comprising:
detecting the second event in response to generating an output via the output element.

13. A method according to claim 10, comprising:
in response to detecting the first event and/or the second event, and a failure to make one or more of the first determination (402), the second determination (408), and the third determination (404):
initiating the first exception procedure (410).

14. A method according to claim 6, comprising:
in response to detecting the second event, and a failure to make at least the third determination (404):
forgoing enabling the main controller (112) to operatively control the motor (106) and movement of the plunger driver at the first period of time; and
initiating the first exception procedure (410).

15. A method according to claim 3, wherein the infusion device includes a display unit; the method comprising:
in response to the second determination (408), corresponding with a first range of orientations of the infusion device, displaying a first display image on the display in accordance with a first display orientation;
in response to the third determination (404), corresponding with a second range of orientations of the infusion device, displaying a second display image on the display in accordance with a second display orientation.

16. A method according to claim 12, comprising:
at the third time, communicating a second message via the first output element and starting a timer with a second time period; wherein the third time is at or prior to the second time and at or after the first time;
in accordance with determination that the second determination is made before the second time period lapses, enabling the main controller (112) to operatively control the motor (106) and movement of the plunger driver at the first period of time;
in accordance with determination that the second determination cannot be made before the second time period lapses, communicating a third message via the output element and forgoing enabling the controller (112) to operatively control the motor (106) and movement of the plunger driver at the first period of time.

17. A method according to claim 1, comprising:
at the first period of time, at a time when the controller is enabled to operatively control the motor and the movement of the plunger driver, performing a priming step including operating the motor to enact that the plunger driver (104) engages the end of the plunger (202) of the syringe (200) and moves the plunger (202) in a longitudinal direction of the syringe (200); wherein at least a volume of air is expelled from the syringe;
wherein the first range of orientations of the infusion device corresponds with an outlet of the syringe pointing upwards.

18. An infusion device, comprising:
a receptacle (120) configured to receive a syringe (200) comprising a plunger (202);
a plunger driver (104) configured to releasably engage an end of the plunger (202) of the syringe (200) and to move the plunger (202) in a longitudinal direction of the syringe (200);
a drivetrain (105), including a motor (106), coupled to move the plunger driver (104);
a controller (112) configured to control travel of the plunger driver (104) in at least a first direction;
an orientation sensor (114), including an accelerometer with a suspended mass, configured to output a first signal including acceleration information and direction information;
wherein the infusion device is configured to perform the method according to claim 1.

19. An infusion device according to claim 18, comprising one or more of:
a first input element connected to the controller for receiving a user input at the input element; and
an output element connected to the controller; wherein the output unit includes one or more of: a display unit, a sound actuator, and a haptic actuator.

20. In some examples, the display unit is a graphical display, e.g. a touch-sensitive display including the first input element. An infusion device according to claim 18, wherein the infusion device is configured to receive a battery; wherein the battery can supply battery power to the controller.
